(19) United States
(12) Patent Application Publication
(10) Pub. No.: US 2012/0238906 A1
(43) Pub. Date: Sep. 20, 2012

(54) LABELED SKIN LESION BIOPSY PUNCH AND USES THEREOF

(75) Inventors: Barbara Ann Gilchrest, Boston, MA (US); Gary Chuang, Boston, MA (US)

(73) Assignee: TRUSTEES OF BOSTON UNIVERSITY, Boston, MA (US)

(21) Appl. No.: 13/383,372

(22) PCT Filed: Jul. 16, 2010
(86) PCT No.: PCT/US2010/042328

§ 371 Date: Apr. 27, 2012

Related U.S. Application Data
(60) Provisional application No. 61/271,003, filed on Jul. 16, 2009.

(51) Int. Cl. A61B 10/02; A61B 10/06

(52) U.S. Cl. 600/567; 600/562; 600/564

(57) ABSTRACT

The present invention relates to a biopsy device, methods, kits and systems for marking the location of a biopsy site in order to later identify the location of a biopsy or surgery in a subject, where the marker is a dye or tattoo which is not visible to the naked eye under normal (white) light, but is visible under UV light. Some embodiments relate to marking a biopsy site for later visualization of the marker.

### CROSS REFERENCE TO RELATED APPLICATIONS

This application claims benefit under 35 U.S.C. 119(e) of U.S. Provisional Patent Application Ser. No: 61/271,003 filed Jul. 16, 2009, the contents of which are incorporated herein in their entirety by reference.

### FIELD OF THE INVENTION

The present invention generally relates to the field of skin marking dyes and systems and methods for their use in the identification prior to, during and after medical procedures, such as the identification of a specific location of a biopsy site. The present invention relates to markers to be employed at biopsy sites to permanently or temporarily mark the site, and to methods and apparatus for applying the permanent or temporary marker. Other aspects of the present invention also relate to methods and devices for marking and defining particular locations in body tissue, particularly human tissue, and more particularly relates to methods and devices for permanently or temporarily defining the location and margins of lesions detected in biopsy cavity walls.

### BACKGROUND OF THE INVENTION

In the U.S., there are more than 1.3 million non-melanoma skin cancers (NMSC) occur each year, which cause significant morbidity and incur high cost to society. Of these, basal cell carcinomas (BCCs) account or about a million and represent the most common malignancy. Squamous cell carcinomas (SCCs) comprise most of the rest of the NMSCs. A suspicious skin lesion needs to be biopsied to confirm the diagnosis of NMSC. The biopsy is done either as a shave or a punch biopsy, in which a small, usually 4mm in diameter part of the suspicious lesion is removed to be sent for pathological diagnosis. Depending on the area of skin involved and delay in the return or follow-up appointment, the biopsy scar may be invisible when the patient returns because the wound had healed well or the scar might be hidden in the background of severe photodamage and prior surgical scars. This creates the possibility of performing surgery on the wrong area or imposing the requirement for additional biopsies.

Certain diagnostic procedures for risk of skin cancer require assessment and monitoring of skin pigmentations and cutaneous marks on the body at different time intervals over a prolonged period of time. For accurate diagnosis and monitoring, it is vital that a physician locate and identify the same skin pigmentation or cutaneous mark repeatedly to identify if there is a cancerous portion of the body and apply appropriate measures such as biopsy excision and/or radiation therapy only to that particular location. Accordingly, some physicians use photography and charts to repeatedly identify the mark of interests. While in other instances, a physician may use a medical tattoos to map out the cancerous region for radiation therapy treatment of those regions. However, these tattoos are visible under ordinary light and thus may be distasteful to many patients. Nevertheless, because of the importance of radiation therapy most patients swallow their reservations and agree to the tattooing.

In particular, the treatment of NMSCs is primary by surgery. Conventional surgical modalities, including excision, electrodessication and curettage, and cryosurgery are the primary method of treatment. The wait time between the time of the biopsy of a NMSC and the surgical appointment may range from 1 week to 3 months or more, at which point the biopsy wound had an opportunity to heal. Also, the physicians performing the biopsy is often not the physician who performs the definited surgery. Thus, there is a grave need for a tool to universally identify the biopsy site between healthcare providers.

The current method of localizing the specific site of biopsy includes digital photography and drawing on the chart. Digital photography relies on the ability of the clinician to capture anatomical landmarks in the photo image. In locations like scalp, back, arms, where visible and unequivocal anatomical landmarks are sparse, digital photography becomes difficult to carry out. Pictorial documentation has traditionally been used, but the advent of electronic medical record system has made pictorial documentation difficult to do. As of yet, Logician, the computerized charts at the Boston Medical Center, for example, cannot accommodate digital photographs. The shortcoming of the above-mentioned systems led the clinician to rely heavily on the patient to identify the area of the biopsy. When the biopsy occurs in area that is difficult to reach, such as back of the ear, top of the scalp and back, the patient may have a hard time identifying the biopsy site. The difficulty is especially more concerning in the elderly population with the highest incidence of NMSC who have difficulty caring for and remembering the location of the biopsy. Moreover, a recent publication (Perlis CS et al., 2006) shows that wrong-site operation is the most common reason for a lawsuit against Mohs surgeons.

Tattoo, or introduction of dermal pigmentation, has been employed in various medical subspecialties for a long time. Tattooing has been employed to mark the area of colon cancer removal, facial prosthesis placement and x-irradiation portals. In addition, micropigmentation is a growing field for pigmentation of skin in procedure such as post-mastectomy areolar reconstruction. Carbon graphite and India ink tattoos have been widely used as a tool for localizing the area treated with radiation. The downside to the carbon graphite and India ink tattoo is that these tattoos are permanent and readily visible. Especially in area of chronic photodamage, black and blue coloration may be mistaken for a lesion concerning for melanoma. Thus, there exists a need in the art for a method to identify a biopsy site without being confused or mistaken as a melanoma.

### SUMMARY OF THE INVENTION

The present invention generally relates to a skin marking biopsy device, where the cutting edges of the biopsy device is coated with a biocompatible dye, e.g., where the dye, e.g., a fluorescent dye, e.g., a UV-dye is invisible under ordinary (white) light, and visible under predetermined wavelengths, such that the cutting edge of the biopsy device can create a tattoo with the dye, e.g., fluorescent dye at the location of the biopsy site on the body, thus ensuring that subsequent definitive surgery is performed at the correct same location.

Accordingly, in some embodiments, the present invention relates to a method, system, kits and devices for producing a tattoo at the location of a biopsy site which is invisible under normal (white) light and is visible under light of a predefined wavelength, e.g., under blacklight. Thus, exposing the tattoo to the predefined wavelength, e.g., blacklight at second timepoint or any subsequent timepoint can be used to locate a biopsy site performed at an earlier timepoint (e.g. a first timepoint).

The use of the tattoo can be used for follow-up monitoring the tissue of the biopsy site, as well as mapping for subsequent radiation therapy and the like.

Accordingly, embodiments of the present invention relate to a method, system, kit, and biopsy device for marking a biopsy site, where the marker is a biocompatible dye, e.g., a fluorescent dye which allows the tattoo, e.g., fluorescent tattoo to blend unobtrusively with the subjects own skin color, permitting marking the biopsy site with a tattoo that is virtually unnoticeable under normal conditions (i.e., in the absence of UV blacklight).

Accordingly, embodiments of the present invention include one or more of the following advantages. As the biopsy device and method and systems enable concurrent delivery of the dye, e.g., fluorescent dye marker at the biopsy location site at the time of the biopsy procedure, it allows for a physician to definitively identify and determine the exact site or location of the biopsy at a later timepoint, e.g., for a diagnostic and/or follow-up assessment by a physician. Additionally, concurrently delivery of the dye, e.g., fluorescent dye marker can be delivered to the biopsy site through the sampling portion of the biopsy device so that the device does not have to be removed before depositing the dye, e.g., fluorescent dye at the biopsy site. Accordingly, another advantage of the present invention relates to being able to mark the location or margins of such a lesion immediately after removing the biopsy tissue mass or sample. In some embodiments, where the biopsy device removes the entire lesion or skin abnormality is removed in its entirety, marking the biopsy site immediately at the same time as the biopsy procedure ensures precise reestablishment of the biopsy location for future identification. The skin marking system and biopsy device allows concurrent marking of the biopsy site at the same time as the tissue sample is removed from the subject, therefore prevents repeated removal of the biopsy device and insertion of a biopsy marking device which may cause unneeded additional discomfort to the patient undergoing the procedure; as well as avoids removal of the biopsy device to be replaced with a marking device, which may introduce an error in placement of the biopsy marker into the desired location; as well as repeated removal and insertion of each of the devices may prolong the duration of the procedure or spread cancer cells etc.

In some embodiments, the dye, e.g., fluorescent dye can be deposited to indicate the circumference of the biopsy site (e.g., at the biopsy cavity walls), and in some embodiments, at opposite the ends of the biopsy site. Additionally, marking a biopsy site with a fluorescent tattoo facilitates non-invasive and continued monitoring of the biopsy site, which enables effective treatment strategies to be devised. For example, a therapeutic device or therapeutic treatment or agent can be guided by and located to a specific biopsy site at the location of the biopsy fluorescent tattoo.

Another advantage is the sociological and psychological advantages to the subject, including the fact that the tattoo is substantially invisible in normal light (white light) avoids having a visible mark which could be an unpleasant or unwanted constant reminder of the biopsy procedure and/or could exasperate a fear of reoccurrence of a cancer in the subject at any time in the future.

In some embodiments, the skin marking biopsy device of the present invention is used to mark and localize a body area of medical interest. In some embodiment, the fluorescent dye is a biocompatible fluorescent dye that is visible under UV illumination, such as, for example, Wood's light and is minimally visible under visible ambient light.

In some embodiments, the dye, e.g., fluorescent dye can be at least partially, or fully biodegradable. When several biopsy samples (e.g., 3-12) are taken, the biopsy device can deposit different colors of dye, e.g., different colors of fluorescent dye to different biopsy locations, so they can be distinguished from one another by the type of fluorescent color used in the dye. For example, in some embodiments, a fluorescent dye can be white, or yellow or orange, or other colors under a defined wavelength, e.g., UV or blacklight, yet substantially invisible under normal (white) light. In some embodiments, a subject can undergo multiple different biopsy, each marked with a different color fluorescent dye, e.g., white, or yellow or orange, or another color, enabling subsequent distinction of each biopsy sites from one another when they are identified and visualized at a later timepoint under the defined wavelength, e.g., e.g., UV or blacklight, e.g., Wood's light.

One aspect of the present invention relates to a tissue marking system comprising combining a biopsy device with a biocompatible dye, wherein the biopsy device applies the biocompatible dye to the biopsy cavity walls or surface of a subject's tissue during a biopsy procedure, and wherein the biocompatible dye is visible under a predetermined wavelength. In some embodiments, the biocompatible dye is a fluorescent dye, for example, a biocompatible dye is not visible or minimally visible under normal (white) light, and is visible under UV light or black light. In some embodiments, the biocompatible dye, e.g., fluorescent dye is visible at under 400 nm wavelength, for example at about 365 nm wavelength.

In some embodiments, the system, method and kits as disclosed herein comprises a biopsy device which comprises a tissue cutting edge, and in some embodiments, the cutting edge is coated with the biocompatible dye.

In some embodiments, the system, method and kits as disclosed herein is used on a biopsy performed on the skin, for example, the tissue which is marked with the system as disclosed herein is skin, such as the dermis. In some embodiments, the system, method and kits as disclosed herein comprises a biopsy device which is a cutaneous biopsy device. Cutaneous biopsy devices are well known in the art, and include, for example, but without limitations, punch biopsy devices, scrape biopsy device, and the like.

In some embodiments, the system, method and kits as disclosed herein comprises a biocompatible dye which is semi-permanent, for example, a biocompatible dye can degrades after a predetermined time such that it is not visible under the predetermined wavelength. In some embodiments, a semi-permanent dye can degrades after at least 6 months, or after at least 12 months, or after at least 2 years.

Another aspect of the present invention relates to a biopsy device, for example, for use in the methods, systems and kits as disclosed herein, wherein the biopsy device comprises at least one tissue cutting edge, wherein at least one tissue cutting edge is coated with a biocompatible dye and wherein the biocompatible dye is reactive under a predefined wavelength. In some embodiments, the biocompatible dye is a fluorescent dye, for example, a dye which is not visible or minimally visible under normal (white) light, but is visible under UV light or black light. In some embodiments, the biocompatible dye is visible at under 400 nm wavelength, or at about 365 nm wavelength. In some embodiments, a biopsy device, for example, for use in the methods, systems and kits as disclosed herein, is a cutaneous biopsy device, such as a cutaneous needle biopsy device, or other well known biopsy devices, for example, but not limited to a needle biopsy device, hookwire biopsy device, photonic needle, clamp, forceps, micro-scissors, punch biopsy device, core biopsy device, razor, scapel blade, suture, shave biopsy device, a cutaneous needle biopsy device.

In some embodiments, a biopsy device, for example, for use in the methods, systems and kits as disclosed herein, is a punch biopsy device, or a shave biopsy device.

In some embodiments, a biopsy device, for example, for use in the methods, systems and kits as disclosed herein, is a disposable biopsy device. In some embodiments, a disposable biopsy device is a scalpel blade, or a flexible blade or a suture.

Another aspect of the present invention relates to a method of determining the site of a biopsy, comprising: (a) using the system as disclosed herein, or the biopsy device as disclosed herein when an initial biopsy is being performed to mark the site of the biopsy (b) locating the biopsy site at a subsequent timepoint by illuminating the skin with the predefined wavelength.

Another aspect of the present invention relates to a method of marking the site of a biopsy, comprising using the system as disclosed herein, or the biopsy device as disclosed herein to mark the biopsy site where a biopsy is being performed, wherein the mark can be detected at subsequent timepoint by illuminating the site of the biopsy with the predefined wavelength.

Another aspect of the present invention relates to a method for identifying the location of a biopsy site wherein the biopsy site was previously marked, comprising illuminating the skin with a predefined wavelength, and wherein the biopsy site was marked at the time of the biopsy procedure using the system as disclosed herein, or the biopsy device as disclosed herein with a marker of the predetermined wavelength. In some embodiments, the biopsy site is illuminated and detected at any timepoint after the initial marking of the biopsy site at the time of the biopsy procedure.

Another aspect of the present invention relates to method of determining the site of a biopsy, for example where one physician can mark the location of the biopsy site at a first time point at the time of the biopsy procedure using the systems and biopsy device as disclosed herein, and then at a second timepoint, e.g., at a follow-up assessment, the same and/or a different physician can identify the location of the biopsy site by illuminating the patients tissue to identify the location of the biopsy site. For example, in some embodiments, a method of determining the site of a biopsy comprises (a) at a first timepoint, using the system as disclosed herein, e.g., using a tissue marking system comprising combining a biopsy device with a biocompatible dye, wherein the biopsy device applies the biocompatible dye to the biopsy cavity walls or surface of a subject's tissue during a biopsy procedure, and wherein the biocompatible dye is visible under a predetermined wavelength, or using a biopsy device as disclosed herein, e.g., biopsy device comprises at least one tissue cutting edge, wherein at least one tissue cutting edge is coated with a biocompatible dye and wherein the biocompatible dye is reactive under a predefined wavelength; and (b) at a second time point, locating the biopsy site at a second timepoint by illuminating the skin with the predefined wavelength. As discussed herein, the marking of the biopsy site during the biopsy procedure, e.g., using the systems and/or biopsy devices as disclosed herein at the first time point can be performed by the same, or a different clinician to the locating the biopsy site at a second time point, by illustrating the skin at the predefined wavelength.

Another aspect of the present invention relates to a method of determining the site of a biopsy, for example where one physician can mark the location of the biopsy site initially (e.g., at a first time point), e.g., when the biopsy procedure is initially being performed using the systems and biopsy device as disclosed herein. Accordingly, one aspect of the present invention relates to a method for marking a biopsy site when the biopsy is being performed (e.g., at first timepoint) using the system or device as disclosed herein, where the mark can be detected at any subsequent timepoint later by illuminating the skin with the predefined wavelength to identify the location of the biopsy site.

Another aspect of the present invention relates to a method for identifying a biopsy site, wherein the biopsy site is identified by illuminating the skin with the predefined wavelength to identify the location of the biopsy site, and wherein the biopsy site was marked at the time of the biopsy procedure using the system or device as disclosed herein.

Another aspect of the present invention relates to a method for identifying a biopsy site at a second timepoint, e.g., such as a follow-up appointment and/or a monitoring assessment of the biopsy site), wherein the biopsy site is identified by illuminating the skin with the predefined wavelength to identify the location of the biopsy site, and wherein the biopsy site was marked at the time of the biopsy procedure (e.g., a first timepoint) using the system and/or device as disclosed herein.

In some embodiments, the time between the first and second timepoint can be anywhere deemed by one of ordinary skill in the art for a follow-up monitoring of a biopsy procedure, for example, the time between the first and second timepoint can be anywhere between about 1 week and about 3 years or more, for example, anywhere between about 1 week, or anywhere between about 1 week and about 1 month, and between about 1 month and about 6 months, and between about 3 months and about 6 months, and between about 6 months to about 1 year, or between about 6 months to 2 years or between about 2 to about 3 years or more than three years. In some embodiments, the first timepoint can be followed up with multiple different second timepoints after the first timepoint, for example, the location of the biopsy site can be located at a third timepoint, or at a fourth timepoint, or at a fifth timepoint, or at a 6 timepoint, or at a seventh timepoint or at any timepoint deemed by one of ordinary skill in the art for following up, and/or monitoring a mark following a biopsy procedure.

Any and all aspects of the methods of the present invention as disclosed herein can use the systems as disclosed herein, (e.g., using a tissue marking system comprising combining a biopsy device with a biocompatible dye, wherein the biopsy device applies the biocompatible dye to the biopsy cavity walls or surface of a subject's tissue during a biopsy procedure, where the biocompatible dye is visible under a predetermined wavelength), and/or can use a biopsy device as disclosed herein (e.g., biopsy device comprises at least one tissue cutting edge, wherein at least one tissue cutting edge is coated with a biocompatible dye and wherein the biocompatible dye is reactive under a predefined wavelength)

Another aspect of the present invention relates to a method of marking a biopsy site comprising; (a) identifying a target area of a subjects skin for a biopsy procedure; (b) using a biopsy device as disclosed herein comprising a biocompatible dye which is reactive at a predetermined wavelength, wherein the biopsy device is inserted into a tissue mass of the subject to be removed by the biopsy procedure; and (c) causing the biopsy device in the tissue mass to deposit the biocompatible dye in a biopsy cavity of the tissue mass as the tissue mass is removed by the biopsy device.

In some embodiments, a biopsy device as disclosed herein for use in the system, methods and kits as disclosed herein comprises a tissue cutting edge. In some embodiments, the tissue cutting edge of the biopsy device is coated with the biocompatible dye, and wherein insertion of the tissue cutting edge of the biopsy device into the tissue mass deposits the biocompatible dye in a biopsy cavity of the tissue mass as the tissue mass is removed by the biopsy device. In some embodiments, when the tissue cutting edge of the biopsy device is inserted into a subjects tissue mass, e.g., skin, the tissue cutting edge deposits a portion, or the entire amount of the biocompatible dye in a biopsy cavity of the tissue mass, e.g., skin, as the tissue mass is removed by the biopsy device, such that an amount of the biocompatible dye remains in the biopsy cavity, and/or on the biopsy cavity walls of the subject after the tissue mass is removed. In such an embodiment, the biocompatible dye remains at the exact location of the biopsy site, and can be detected at a later timepoint, e.g., a second or subsequent timepoint by illuminating with a predefined wavelength to visualize the biocompatible dye as disclosed herein.

Another aspect of the present invention relates to a kit for use in the methods and systems as disclosed herein, wherein the kit comprises a container which comprises an amount of biocompatible dye for use in combination with the biopsy device, e.g., for example, coating a tissue cutting edge of a biopsy device. In some embodiments, the amount of amount of biocompatible dye is sufficient for coating the tissue cutting surface at least one, or at least 2, or at least 3 or at least 4, or at least 5, or a least 6, or at least 7, or at least between 7-10, or at least between 5-15, or at least between 10-20 or more than 20 biopsy devices. In some embodiments, the kit further comprises a biopsy device, for example, a disposable biopsy device or a disposable attachment for a non-disposable biopsy device. In some embodiments, a disposable attachment for a non-disposable biopsy device comprises at least one tissue cutting edge. In some embodiments, a kit can further comprise an apparatus to aid coating at least one tissue cutting edge of a biopsy device with an amount of the biocompatible dye.

In some embodiments, the kit comprises a biocompatible dye which is a fluorescent dye, for example, a biocompatible dye which is not visible, or is minimally visible under normal (white) light, for example but not limited to a biocompatible dye which is visible under UV light or black light. In some embodiments, a biocompatible dye of the kit is visible at under about 400 nm wavelength, or is visible at about 365 nm wavelength, or is visible at below about 365 nm wavelength.

Another aspect of the present invention relates to a kit for use in the methods and systems as disclosed herein, wherein the kit comprises at least one disposable biopsy device, wherein the disposable biopsy device has at least one tissue cutting edge, and wherein the at least one tissue cutting edge is coated with a biocompatible dye which is reactive under a predetermined wavelength. In some embodiments, the kit comprises at least about 5 disposable biopsy devices, or at least about 10 disposable biopsy devices, or at least about 15 disposable biopsy devices, or at least about 20 disposable biopsy devices, or more than 20 disposable biopsy devices. In some embodiments, each disposable device can be individually packaged in sterile packaging. In some embodiments, more than one disposable biopsy device is packaged in sterile packaging, e.g., at least 2, or at least 5, or at least about 10 or more disposable biopsy devices packaged together in sterile packaging.

In some embodiments, a kit can comprise a disposable biopsy device which is a disposable attachment comprising at least one tissue cutting edge which can be attached to a non-disposable biopsy device. In some embodiments, a disposable biopsy device of the kit can comprise at least one tissue cutting edge selected from the group consisting of: scalpel blade, flexible blade, sutures and the like. In some embodiments, a disposable attachment which attaches to a non-disposable biopsy device (e.g., a non-disposable handle of a biopsy device) can comprise at least one tissue cutting edge is selected from the group consisting of: scalpel blade, flexible blade, sutures and the like.

In some embodiments, the biopsy device and methods as disclosed herein can be used for marking the specific location of a biopsy site, for example, where it is desirable to identify the location of the biopsy site at a future timepoint, e.g., at a follow-up appointment, and where it is desirable to have a mark of the site which is not visible, or is minimally visible under normal light, e.g., white light.

In some embodiments, the tissue marking system as disclosed herein, e.g., a skin-marking system as disclosed herein is useful for being able to identify and determine the location of the site of a biopsy procedure which as been performed previously on a subject, in particular, where it is desirable to identify the location of the biopsy site at a future timepoint, e.g., at a follow-up appointment, and where it is desirable to have a mark of the site which is not visible, or is minimally visible under normal light, e.g., white light.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIGS. 1A-1D show examples of tissue marked by the fluorescent dye under different light conditions. FIG. 1A shows 8 skin tissue samples after washing with PBS for 2 days with white fluorescent dye (white), yellow and orange fluorescent dye and two samples not labeled (neg) under normal (white) light. No visible detection of the fluorescent dyes. FIG. 1B shows 8 skin tissue samples after 2 days of washing with white fluorescent dye (white), yellow and orange fluorescent dye and two samples not labeled (neg) under UV light from a tissue culture hood. The fluorescent dye for the yellow and orange fluorescent dye is visible under UV light. FIG. 1C shows the fluorescent dye is clearly visible for the white, yellow and orange fluorescent dyes after 2 days of washing under blacklight (Woods lamp, e.g. UV light). FIG. 1D shows the fluorescent dye is clearly visible for the white, yellow and orange fluorescent dyes after 5 days of washing under blacklight (Woods lamp, e.g. UV light). No marker is visible in the negative (neg) controls under UV hood light, or blacklight at either 2 or 5-days after biopsy excision (FIGS. 1B-1D).

FIGS. 2A-2B show representative images of hematoxylin and eosin staining (H&E) and fluorescent images of the negative control (no fluorescent dye) after 5 days of washing with PBS. FIG. 2A shows hematoxylin and eosin staining (H&E) and FIG. 2B shows fluorescent image of the negative treated skin sample, showing no immunofluorescence under blacklight (e.g., UV light).

FIGS. 3A-3B show representative images of hematoxylin and eosin staining (H&E) and fluorescent images of the skin sample tattooed with the orange fluorescent dye after 2 days of washing with PBS. FIG. 3A shows hematoxylin and eosin staining (H&E) and FIG. 3B shows fluorescent image of the skin sample tattooed with the orange fluorescent dye, showing no visulation under normal (white) white, but clear fluorescence under blacklight (e.g., UV light) at the edge of the tissue sample (e.g., at the biopsy cavity walls).

FIGS. 4A-4B show representative images of hematoxylin and eosin staining (H&E) and fluorescent images of the skin sample tattooed with the orange fluorescent dye after 5 days of washing with PBS. FIG. 4A shows hematoxylin and eosin staining (H&E) and FIG. 4B shows fluorescent image of the skin sample tattooed with the orange fluorescent dye, showing no visulation under normal (white) white, but clear fluorescence under blacklight (e.g., UV light) at the edge of the tissue sample (e.g., at the biopsy cavity walls) after 5 days of washing.

FIGS. 5A-5B, similar to FIGS. 4A-4B, are additioanal representative images of hematoxylin and eosin staining (H&E) and fluorescent images of the skin sample tattooed with the orange fluorescent dye after 5 days of washing with PBS. FIG. 5A shows hematoxylin and eosin staining (H&E) and FIG. 5B shows fluorescent image of the skin sample tattooed with the orange fluorescent dye, showing no visulation under normal (white) white, but clear fluorescence under blacklight (e.g., UV light) at the edge of the tissue sample (e.g., at the biopsy cavity walls) after 5 days of washing.

FIG. 6A-6F shows a case study of use of one embodiments of the invention to mark the site of a biopsy with a fluorescent tattoo and follow-up three months later. FIG. 6A shows a patient with multiple BCC (basal cell carcinomas) on his back. A lesion in the middle of the subjects back was suspected to be recurrent BCC s/p multiple LN2 and ED&C by FP. The lesion was removed by the methods and systems as disclosed herein, as well as a biopsy device as disclosed herein where the fluorescent dye was coated in the surface of the biopsy cutting surface. FIG. 6B shows an image of the biopsy site with the fluorescent dye marking the biopsy site under blacklight, e.g., Woods lamp on the day of the biopsy (Bx) procedure. FIG. 6C shows an image of the region of the lesion on the subjects back at a 3-month physician follow-up under normal (white) light, where the fluorescent tattoo marker is not visible. FIG. 6D shows an image of the region of the lesion on the subjects back at a 3-month physician follow-up under blacklight, e.g., U.V. light using a Wood's lamp, where the fluorescent tattoo marker is clearly visible, enabling the physician to clearly identify the precise location of the biopsy site at the 3-month follow-up assessment. FIG. 6E is a high magnificent image of the fluorescent tattoo marker shown in FIG. 6D. FIG. 6F shows an image of the subjects back at a 6-month follow-up with the physician, where the patient identified the wrong lesion as the location which was biopsied 6-months earlier, where the correct biopsy site was identified by the fluorescent tattoo marker visible under UV light, but not visible under normal (white) light. The arrow labeled (a) is the correct location of the biopsy site as identified by the fluorescent tattoo under UV light. However, the patient identified an incorrect location shown by the arrow labeled by (b) that the patient thought was the site of the biopsy. The patient did not experience any symptoms or side effects with the fluorescent tattoo.

### DETAILED DESCRIPTION OF THE INVENTION

The present invention generally relates to a biopsy marking device and skin-marking system for the percutaneous placement of a marker at a biopsy site in a tissue mass to facilitate subsequent determination of the location of the biopsy site. In particular, the present invention relates to use of a marker dye which is not visible under ambient light conditions, but is visible under UV light or light with wavelegths less than about 400 nm.

The present invention, relates in part to the combination of a biopsy punch device used with a bio-compatible, yet permanent dye, such as, for example a tattoo dye, coating the cutting edge of the punch device. One method of production of a biopsy device as disclosed herein is to apply the fluorescent dye by itself or encapsulated in bio-compatible material such as polymethymethacrylate, and then waiting for the tattoo to dry in the cutting edge of the biopsy punch. As a part of the coating process, it is possible to embed an electrocharged particle such as a cation or anion during the formation of the tattoo or the bio-compatible capsule that will facilitate adhesion to the metal surfaces of the cutting edge. During the biopsy process, the biopsy punch device will impart the tattoo, which will adhere to the skin edge and mark the location of the biopsy.

Alternatively, it is possible that the UV fluorescent tattoo by itself or coated in a bio-compatible material such as polymethylmethacrylate can be incorporated into the manufacture of metallic cutting edge of the biopsy punch, thus eliminating the extra step of tattoo-coating.

In some embodiments, the dye could be in a packet to coat the biopsy device by the practitioner at the time of use. Any permutation of packaging to contain the dye can be envisaged.

Accordingly, in some embodiments, the present invention relates to a method and device which allows one to localize skin for surgical removal of skin cancer through the use of the UV-fluorescent tattoo. As mentioned herein, the UV-fluorescent tattoo is not visible to the eye under visible light and becomes visible under the Wood's UV lamp. Accordingly, the inventors have developed a method for generating a tattoo at the time of a biopsy which allows for secure localization of the NMSC and has the potential for easily incorporated into the routine practice of biopsy to allow for unequivocal identification of biopsy site. However, the invention can be used to localize other area of body of medical interest, and is not necessary limited to marking of the skin. For example, it can be used to mark the surface of any tissue of a subject, for example a surface of a tissue identified to have risk of cancer, or a surface of a tissue in which tissue has been removed by way of a biopsy.

In some embodiments, the present invention, such as the biopsy device and marking system as disclosed herein is advantageous over other techniques as it allows the physician to accurately position and deploy the fluorescent dye marker at the site of a biopsy at the time of the biopsy procedure (e.g. simultaneously with the biopsy procedure). This provides several advantages to the physician in diagnosis and management of tissue and skin abnormalities, such as a means of localization of a tissue or skin abnormality for follow-up surgical treatment, and a means of tissue abnormality site identification for purposes of ongoing diagnostic follow-up. It may also prevent inadvertent repeat biopsy of a lesion if the patient were to move or if adequate records did not follow the patient, or if the patient is no longer able to identify the site of the biopsy procedure.

Additionally, the present invention of the biopsy device, methods and systems as disclosed herein also has advantages in that it represents a less traumatic means for tissue marking and skin abnormality marking and has a reduced procedural duration relative to the standard open surgical method, where marking of the biopsy site is not performed at the same time as the biopsy tissue extraction, therefore increasing the time for the surgical intervention, or systems where the marking is visible under normal light conditions, such that the marking is a constant reminder to the subject and the public that the subject has had a biopsy has, or has had a risk of cancer. Accordingly, as the present invention relates to methods, devices and systems to identify and locate a biopsy site using a non-visible marker under normal light conditions, but which is visible under UV light, the present invention has significant cosmetic benefit and physiological advantages to the subject which has undergone a biopsy procedure.

### Biopsy Procedure:

A biopsy device as disclosed herein can be used to perform an open or percutaneous biopsy technique. Open biopsy removes the entire mass (excisional biopsy) or a part of the mass (incisional biopsy). Percutaneous biopsy on the other hand is usually done with a needle-like instrument and may be either a fine needle aspiration (FNA) or a core biopsy. In FNA biopsy, very small needles are used to obtain individual cells or clusters of cells for cytologic examination. The cells may be prepared such as in a Papanicolaou (Pap) smear. In core biopsy, as the term suggests, a core or fragment of tissue is obtained for histologic examination, which may be done via a frozen section or paraffin section. The chief difference between FNA and core biopsy is the size of the tissue sample taken. A real time or near real time imaging system having stereoscopic capabilities, such as the stereotactic guidance system described in U.S. Pat. No. 5,240,011, can be employed to guide the extraction instrument to the lesion if the lesion is in an internal tissue or cavity of the subject. Advantageous methods and devices for performing core biopsies are described in U.S. Pat. No. 5,526,822, which is incorporated herein in its entirety by reference.

In some embodiments, a marking device as disclosed herein places the dye, e.g., fluorescent dye at the edge of the biopsy cavity, and thus has the obvious advantage of marking the circumference of a biopsy cavity, thus allowing a physician to identify the exact tissue which was removed in the biopsy procedure.

### Biopsy Device

One aspect of the present invention relates to a surgical device or biopsy device, such as a biopsy needle for excising a tissue sample, e.g., a tissue sample which comprises, or is suspected to comprise cancerous tissue, or is identified by regions indicative of cancer, e.g., skin cancer and melanoma, or other diagnosed types of pathological tissue anomalies, benign tumors (such as e.g. small and large skin pigmentation regions or moles) and malignant, metastasic carcinomas etc. In some embodiments, the surgical device, or biopsy needle is coated on the tissue cutting surface, e.g., the surface which contacts the tissue to removed, with a biocompatible dye (e.g., a fluorescent dye which is in the visible or near-infrared spectrum of light). The tissue cutting edge can contact a tumor site or other pathological tissue anomaly for its removal from the subject. As the tissue cutting edge of the surgical device or biopsy device, (e.g., needle or core biopsy device) contacts the subjects tissue and removes a tissue sample, e.g., tissue mass from the subject, the tissue cutting edge will leave some of the biocompatible dye, e.g., a fluorescent dye on the tissue at the boundary where the tissue sample was removed from the subject, thus marking the location of the biopsy. In some embodiments, when the tissue cutting surface is inserted into the tissue to be removed, it deposits some biocompatible dye in the biopsy cavity when the tissue mass is removed, and/or can deposit some of the biocompatible dye on the biopsy cavity wall where the tissue mass is removed. In some embodiments, where the biocompatible dye is permanent, it helps a surgeon to find the boundary of the biopsy lesion at any date in the future. In some embodiments, where the biocompatible dye is semi-permanent, it helps a surgeon to find the boundary of the biopsy lesion at any dates in the future within the predetermined time before the semi-permanent dye degrades.

During the production of the biopsy equipment such as biopsy punch or blade, the outer surface of the sharp edge will be inoculated with a drop of the sterile UV-fluorescent dye. Alternatively, the biopsy punch or blade (for shave biopsies) can be manufactured with the dye already on a tissue cutting surface. The biopsy equipment will be applied to the intended site of biopsy. The biopsy will then be performed as per standard of care, innoculating or placing the biocompatible dye into the tissue mass, e.g., skin or dermis.

In some embodiments, the practice of innocoluating a fluorescent dye, which is only visible under UV light to the biopsy equipments as a way to localize the biopsy site at a later time point has not been previously described or been reported in the prior-art. Previous markings have been done either after the biopsy procedure, or using a permanent dye which is visible under normal ambient light conditions, e.g. using india ink or carbon-based tattoo dyes. Accordingly, the inventor's discovery of simultaneously marking the site of the biopsy at the same time as performing the biopsy has not been performed in the prior art.

For example, a needle biopsy device has been reported which includes a two-stage actuation mechanism in which the cutting needle and cannula are advanced in timed sequence under spring actuation is described in Bates, U.S. Pat. No. 4,958,625, the entire contents of which are incorporated herein by reference. In some embodiments, the biocompatible dye, e.g., fluorescent dye may be introduced into a biopsy cavity by various suitable percutaneous access biopsy devices, e.g., as described in U.S. Pat. No. 6,356,782 and U.S. Pat. No. 6,371,904 to Sirimanne et al., and U.S. Publication No. 2003/0050571 to Zarins et al which are herein incorporate by reference in their entireties. Other biopsy devices which are useful in the methods, kits, systems as disclosed herein are disclosed in U.S. patent applications 2010/0013920, 2009/054806, 2006/0111646, 2008/0188768,2006/0025795, 2004/0049126, and International Patent application WO 2009/050667, which are incorporated herein in their entireties by reference.

In some embodiments, a biopsy device as disclosed herein, which in some embodiments, can be coated on its cutting surface with a biocompatible dye, e.g., a fluorescent dye, is chosen to match the desired size of the biopsy sample to be taken from the subject.

A number of procedures and devices for marking and locating particular tissue locations are known in the prior art, however these do not disclose simultaneously marking the biopsy site with a dye which is only visible under UV light. For example, location wire guides, such as that described in U.S. Pat. No. 5,221,269 to Miller et al, are well known for locating lesions, particularly in the breast. The device described by Miller comprises a tubular introducer needle and an attached wire guide, which has at its distal end a helical coil configuration for locking into position about the targeted lesion. The needle is introduced into the breast and guided to the lesion site by an imaging system of a known type, for example, x-ray, ultrasound, or magnetic resonance imaging (MRI), at which time the helical coil at the distal end is deployed about the lesion. Then, the needle may be removed from the wire guide, which remains in a locked position distally about the lesion for guiding a surgeon down the wire to the lesion site during subsequent surgery. While such a location system is effective, it is obviously intended and designed to be only temporary, and is removed once the surgery or other procedure has been completed.

Other devices are known for marking external regions of a patients skin. For example, U.S. Pat. No. 5,192,270 to Carswell, Jr. discloses a syringe which dispenses a colorant to give a visual indication on the surface of the skin of the point at which an injection has or will be given. Similarly, U.S. Pat. No. 5,147,307 to Gluck discloses a device which has patterning elements for impressing a temporary mark in a patient's skin, for guiding the location of an injection or the like. It is also known to tape or otherwise adhere a small metallic marker, e.g. a 3 millimeter diameter lead sphere, on the skin of a human breast in order to delineate the location of skin calcifications (see Homer et al, The Geographic Cluster of Microcalcifications of the Breast, Surgery, Gynecology, & Obstetrics, December 1985). Obviously, however, none of these approaches discuss or mention the use of fluorescent dyes, or other dyes which are not visible under normal light (e.g., ambient light) are visible under UV light for marking and delineating biopsy locations, both cutaneous and internal tissue abnormalities, such as lesions or tumors.

Still another approach for marking potential lesions and tumors of the breast is described in U.S. Pat. No. 4,080,959. In the described procedure, the skin of the portion of the body to be evaluated, such as the breasts, is coated with a heat sensitive color-responsive chemical, after which that portion of the body is heated with penetrating radiation such as diathermy. Then, the coated body portion is scanned for color changes which would indicate hot spots beneath the skin surface. These so-called hot spots may represent a tumor or lesion, which does not dissipate heat as rapidly because of its relatively poor blood circulation (about 1/20 of the blood flow through normal body tissue). This method, of course, functions as a temporary diagnostic tool, rather than a permanent means for delineating the location of a tumor or lesion.

In some embodiments, the biopsy device as disclosed herein is a fine needle aspiration biopsy device, where a small sample of cells is drawn by a thin needle from the lump or area of suspect tissue. If the suspect area or lump cannot be easily felt, non-invasive imaging may be used to help the doctor guide the needle into the right area. In some embodiments, the biopsy device is a core biopsy device, which includes devices for performing a core biopsy, which is similar to a fine needle aspiration biopsy, except that a larger needle is used. Under a local anesthetic, the doctor makes a very small incision in the patient's skin and removes several narrow sections of tissue from the suspect area of tissue through the same incision. The core biopsy device provides tissue mass biopsy sample rather than just individual cells, thus making it easier for the pathologist to identify any abnormalities.

In some embodiments, the biopsy device is a vacuum-assisted biopsy device, where a vacuum-assisted biopsy is performed through the skin and may rely upon ultrasound or stereotactic guidance to determine the location of a suspect area of tissue. Two commonly used vacuum-assisted biopsy systems include are MAMMOTOMETM supplied by Johnson & Johnson Ethicon Endo-surgery or MIBB™ supplied by Tyco International. Examples of such devices may be found in U.S. Pat. No. 5,526,822 entitled “Methods and Apparatus for Automated Biopsy and Collection of Soft Tissue,” U.S. Pat. No. 5,649,547 entitled “Methods and Devices for Automated Biopsy and Collection,” U.S. Pat. No. 6,142,955 entitled “Biopsy Apparatus and Method” and U.S. Pat. No. 6,019,733 entitled “Biopsy Apparatus and Method” the entirety of each of which is incorporated by reference herein. Such biopsy devices are useful in the methods, kits and systems as disclosed herein and can optionally include a probe that is inserted through the skin and is usually adapted to provide a vacuum to assist in obtaining the biopsy sample.

In some embodiments, the biopsy device comprises an attached syringe (e.g., a single-use syringe) which can be filled with the dye, e.g., fluorescent dye according to the invention or, alternatively, the syringe may be part of a kit of the present invention which is a pre-loaded with the dye, e.g., fluorescent dye (e.g., in a port kit including a single use safety needle). The delivery of the dye, e.g., fluorescent dye at the site of the biopsy by the biopsy device can vary on a case to case basis.

In some embodiments, a biopsy device for use in the methods, systems and kits as disclosed herein comprises a biocompatible dye, e.g., a fluorescent dye which is attached to the biopsy device such that the biocompatible dye, e.g., a fluorescent dye is deposited in the biopsy cavity, or on the biopsy cavity as the biopsy tissue mass is removed from the subject. In some embodiments, the biocompatible dye, e.g., a fluorescent dye is used to coat a cutting surface of the biopsy device. In some embodiments, a biocompatible dye, e.g., a fluorescent dye is present in an element, e.g., a tube or cannula attached to the biopsy device, such that the biocompatible dye, e.g., a fluorescent dye is deposited in the biopsy cavity, or on the biopsy cavity walls when a tissue mass is removed by the biopsy device. In some embodiments, the biopsy device is guided to its site by the aid of a visualization device, such as an imaging system, an endoscope, or the like.

For example, a biopsy device can be adapted to both penetrate tissue and to contain a cutting member (e.g., a tissue cutting edge) which facilitates the removal of the biopsy sample, where the cutting edge is coated with the biocompatible dye, e.g., fluorescent dye. In some embodiments, the cutting member can contain an aperture for receiving the tissue mass to be removed from the subject for the biopsy. Once inserted through the skin, the cutting member of the biopsy device can align with suspect tissue, and once the tissue is in the aperture, the cutting member can actuates to capture a tissue mass sample for the biopsy. In some embodiments, a biopsy device of the present invention can be adapted such that the cutting member and aperture rotate (e.g., via manipulation by the handler) with respect to the tissue mass to excise the tissue mass from the subject and create a biopsy cavity with a biopsy cavity wall. As the cutting member and aperture is rotating, the biocompatible dye, e.g., fluorescent dye on the surface of the cutting member is deposited on the biopsy cavity wall and/or in the biopsy cavity.

Accordingly, the biopsy devices as disclosed herein removes tissue from a subject to create at least one tissue cavity, where the dye, e.g., fluorescent dye is deposited after removal of the biopsy sample. In some embodiments, the biopsy device comprises a member, which may comprise a tube, such as a needle, cannula, or trocar, of any known type for delivering the dye, e.g., fluorescent dye to the location of the biopsy site of the subjects. In some embodiments, the biopsy device comprises a biopsy needle or biopsy gun, or core biopsy device such as is often used to extract tissue for examination in a biopsy procedure, is used in conjunction with the biocompatible dye, e.g., fluorescent dye as disclosed herein.

In some embodiments, the biopsy device deposits the biocompatible dye, e.g., fluorescent dye on the cavity wall of the biopsy when the tissue has been removed, thus depositing the dye at the circumference of the biopsy site. In other embodiments, the biopsy device deposits the dye, e.g., fluorescent dye to indicate mapping for cancer radiation therapy, where the biopsy device deposits the dye, e.g., fluorescent dye at more than one location, e.g., a series of locations that may form a substantially solid line spanning the length of the required treatment area, wherein the thickness of the substantially solid line may vary according to the thickness of the required treatment area.

In some embodiments, a biopsy device can deposit the dye, e.g., fluorescent dye in a pattern matching the size and/or anatomy of the target site within the body. For example, a physician can use a biopsy device as disclosed herein, coated on its cutting surfaces with a dye, e.g., fluorescent dye to deposit the dye at multiple locations, and/or multiple punctures at predetermined marginal distances wherein multiple punctures may correlate to the size and anatomy of the target site within the body.

In some embodiments, the biopsy device deposits the dye, e.g., fluorescent dye into a dermis of the skin, for example, but not limited to, at approximately 4.25-5 mm below the skin.

### Dyes and Fluorescent Dye Pigments

In some embodiments, the dye of for use in the methods, systems and devices as disclosed herein include fluorescent pigments, for example, pigments which are visible only when illuminated with ultraviolet or infrared light, or in alternative embodiments, pigments which are phosphorescent, e.g., “glow-in-the-dark” pigments, which emit light for a period of time after being illuminated. In some embodiments, dyes, e.g., fluorescent or phosphorescent dyes can be retained in the dermis by entrapment, encasement, incorporation, complexing, or encapsulation by pigment vehicles to produce tattoo inks which fluoresce or phosphoresce, respectively.

In some embodiments, the fluorescent dye, e.g. is a fluorescent contrast agent such as one that is invisible under visible light and only visible under black light, e.g., a Wood's lamp (long-wave ultraviolet light), or an alternative UV light source that that is readily available in a dermatologist's office.

Blacklight is commonly known to persons of ordinary skill in the art. A blacklight for use in relation with the present invention may be formed in the same fashion as normal fluorescent lights except that only one phosphor is used and the normally clear glass envelope of the fluorescent bulb may be replaced by Wood's Glass, a nickel-oxide doped glass which blocks all visible light above approximately 400 nanometers. Specifically, the intensity of the tattoo of the present invention may peak in the range of approximately 350-405 nm and, more particularly, in the range of 350-370 nm, wherein the location of the intensity peak is dependent on the type of glass used in the blacklight bulb, as those skilled in the art will understand. Accordingly, in some embodiments at light wavelengths within the optical spectrum but outside of the blacklight peak range, e.g., the tattoo of the present invention remains invisible.

In some embodiments, the terms “dye” and “pigment” are interchangeable with respect to preparing the dyes or tattoo inks as disclosed herein. As disclosed herein below, the dyes or pigment vehicles as disclosed herein can be formulated so that they remain indefinitely in the dermis, or in alternative embodiments they can spontaneously disappear after a predetermined period of time, or they can be caused to disappear by imposition of an exogenous force.

In some embodiments, a dye useful in the methods, systems and devices as disclosed herein, e.g., fluorescent dyes can be formulated to resist spontaneous elimination from the dermis by virtue of their inherent physical characteristics, e.g., they are too large to be spontaneously eliminated; or an anchoring system anchors the dye pigment to the surrounding dermal tissue, e.g., by chemical bonding or by encapsulation into dermal cells.

In some embodiments, the dye useful in the methods, system and devices as disclosed herein is a carbon nanotube, where the nanotubes fluoresce, which can be detected by shining near-infrared light on them, as disclosed in U.S. patent application 2009/0251693, U.S. Patent which is incorporated herein in its entirety by reference. One advantage of this type of pigment in the dye is that unlike some fluorescent molecules, carbon nanotubes are not destroyed by light exposure, and their intensity will not change regardless of the level of light exposure they have received.

Alternatively, in some embodiments, a dye useful in the methods, systems and devices as disclosed herein, e.g., fluorescent dyes can be formulated as pigment/vehicle complexes spontaneously disappear, such as by bioabsorption, bioerosion, or biodegradation, after a predetermined period of time (e.g., semi-permanent tattoos), as disclosed in U.S. Pat. No. 6,013,122 which is incorporated herein in its entirety by reference.

In some embodiments of the present invention, the dye can incorporate a filler that is reactive to ultrasonic waves. For example, a filler may be used along with the dye molecules, e.g., fluorescent dye in a microsphere or, in alternative embodiments, can be used in place of the dye, e.g., fluorescent molecules. In such an embodiment, a clinician can view and identify the location of the tattoo, e.g., fluorescent tattoo by passing a wand of an ultrasound machine over the tattooed portion of skin, as those skilled in the art will understand. In some embodiments, a filler material may comprise microspheres or nanospheres with air bubble cores so that the air bubble core will resonate with the ultrasonic energy. Alternatively, in some embodiments, microspheres can comprise another gas filler such as, but not limited to, a perfluorocarbon compound, nitrogen, xenon, argon, helium, nitrous oxide, carbon dioxide.

In some embodiments, to enhance detection by ultrasound, the fluorescent dye can be coated with an echogenic substance. One such substance is the ECHO-COAT.RTM. coating (STS Biopolymers, Henrietta, N.Y.). Echogenic coatings provide the coated marker element with an acoustically reflective interface and a large acoustical impedence differential.

In yet another embodiment of the present invention, the PMMA microsphere 110 may be filled with standard tattoo dye in addition to the fluorescent dye so that all or portions of the tattoo may be visible under white light and UV blacklight.

### Fluorescent Dye Pigment Vehicles

In some embodiments, dyes, e.g., fluorescent dyes are delivered in a vehicle, e.g., a pigment vehicle, e.g., vehicles which encapsulate, entrap, encase, complex, or otherwise incorporate the dye pigments, e.g., fluorescent dyes. In some embodiments, pigment vehicles are biologically tolerated and form color-carrying particles that possess specific characteristics necessary for the type of tattoo ink desired. For permanent tattoos, the pigment vehicles are designed to remain indefinitely in the dermis to prevent the pigment/vehicle complex from being readily eliminated from the dermis. The pigment vehicles resist spontaneous elimination from the dermis by the nature of their inherent physical characteristics (e.g., large size), by immunoprotection (e.g., “stealth” technology using polyethylene glycol incorporation), or by an anchoring system which anchors the vehicle to the dermal tissue (e.g., chemical bonding or encapsulation into dermal cells). These pigment/vehicle complexes are used to form a tattoo ink which can be used in the methods, system and devices as disclosed herein, e.g., in methods and systems to mark the location of the biopsy site, as well as being coated onto a biopsy instrument for simultaneous marking of the site at the same time the tissue biopsy is being performed.

In some embodiments, the UV-fluorescent dye can be made with pure dye or alternatively captured within small bio-compatible pigment vehicle such as polymethymethacrylate (PMMA), which prevents degradation and development of cutaneous sensitivity to the UV-fluorescent dye. PMMA is FDA approved as component of as an orthopedic prosthesis and dermal injection filler. Communication with the FDA has indicated that there is no report of adverse reaction to the PMMA-captured UV-fluorescent tattoo. The FDA also states that, while they do monitor for reports of adverse reaction to tattoos, tattoo dyes are not subject to FDA approval for commercial marketing.

In some embodiments, a biocompatible dye, e.g., fluorescent dye is contained in a vehicle, e.g., a pigment vehicle such as polymethylmethacrylate (PMMA) microsphere. PMMA is used to embed foreign materials in orthopedic and dental prosthesis, and also used in soft tissue augmentation for lipodystrophy. PMMA has not been reported to produce hypersensitivity or allergic reaction. PMMA can be manufactured as an injectable dermal filler: ArteFill and ArteColl (Artes Medical).

In some embodiments, a biocompatible dye, e.g., a fluorescent dye according to the present invention employs blacklight visible ink embedded within polymethylmethacrylate (“PMMA”) microspheres. In some embodiments, the a biocompatible dye can optionally comprise a color dye molecules which can be embedded within the polymethylmethacrylate (“PMMA”) microspheres. Such a color is desirable when you wish match the color dye with the subjects skin or for other reasons. In some embodiments of the present invention, the microspheres may be composed of a polymer material. As commonly known in the art, PMMA microspheres are approximately 4-5 times as large as a human red blood cell. Due to its encasement in the PMMA microspheres, which essentially serves as a shield for the a biocompatible dye, e.g., a fluorescent dye, the fluorescent dye molecules of the present invention do not come into direct contact with the skin of a patient. Those skilled in the art will understand that nanospheres may be substituted for the described microspheres without departing from the scope of the invention and, as used herein, the term microspheres encompasses nanospheres as well.

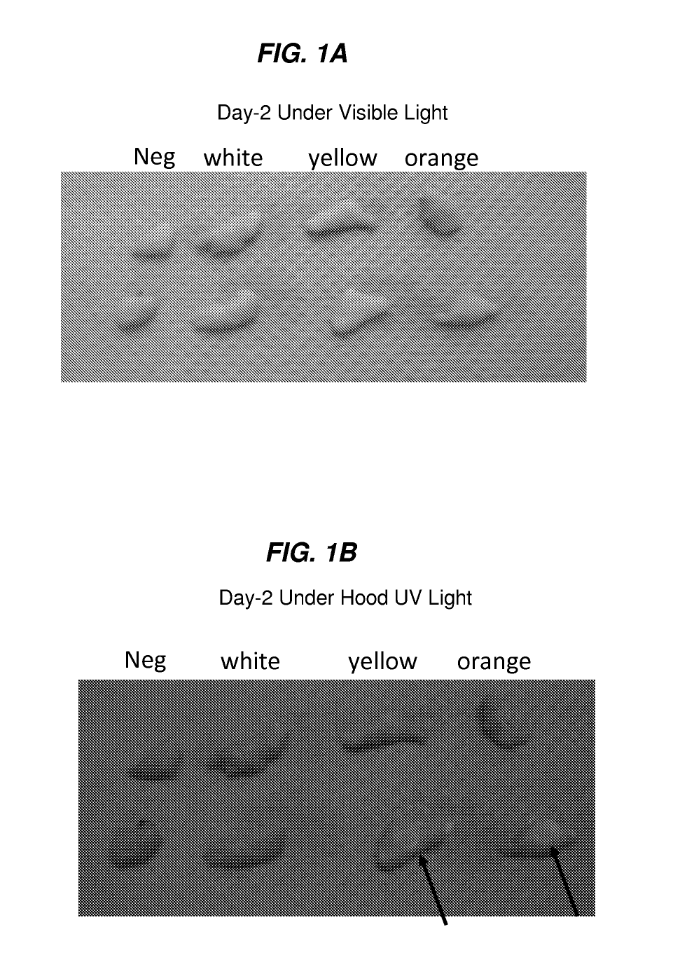

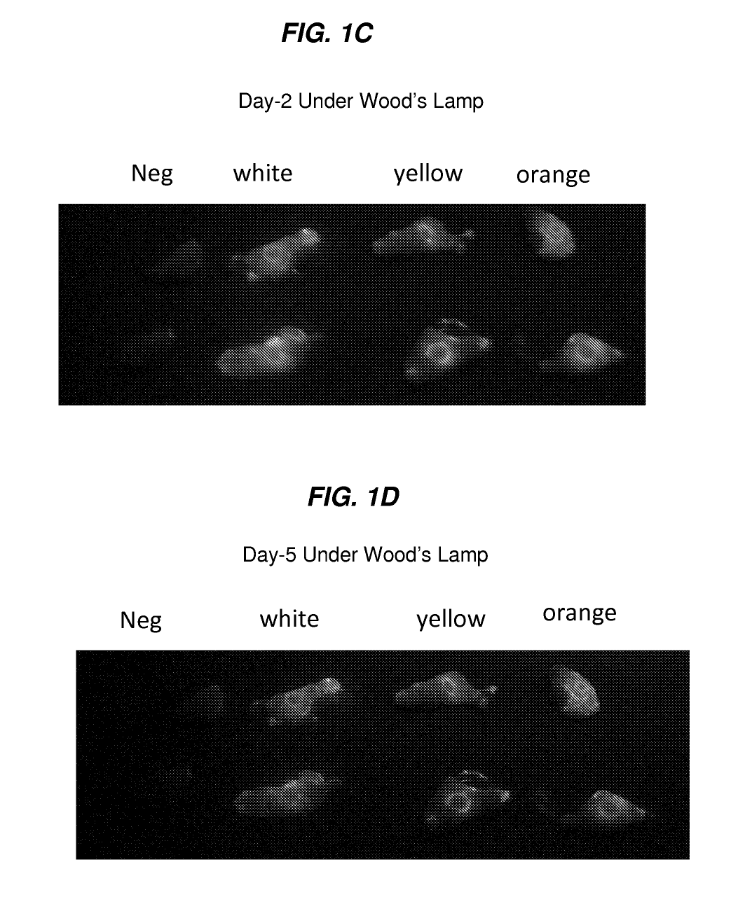

In some embodiments, a biocompatible dye, e.g., a fluorescent dye or pigment molecules can be loaded in the PMMA microspheres, where the dye is a fluorescent dye. Alternatively, in some embodiments, the base of the PMMA microsphere can be substantially the same color as a skin color of a subject. As shown in FIG. 1A, the biocompatible dye, e.g., a fluorescent dye tattoo is not visible under normal (white light), however, as shown in FIGS. 1B and 1D and 1D the fluorescent dye of the tattoo, when viewed under an ultraviolet (“UV”) light from a tissue culture hood (FIG. 1B), or under Wood's blacklight (FIG. 1C, 1D) exhibits an intense fluorescence due to phosphors within the dye which convert energy from the UV radiation into visible light. In some embodiments, the combination of the two components in the color dye molecules results in a colored tattoo that is the same pigmentation, or only slightly darker, than the subjects skin and is only visible when exposed to blacklight from, for example, a UV blacklight.

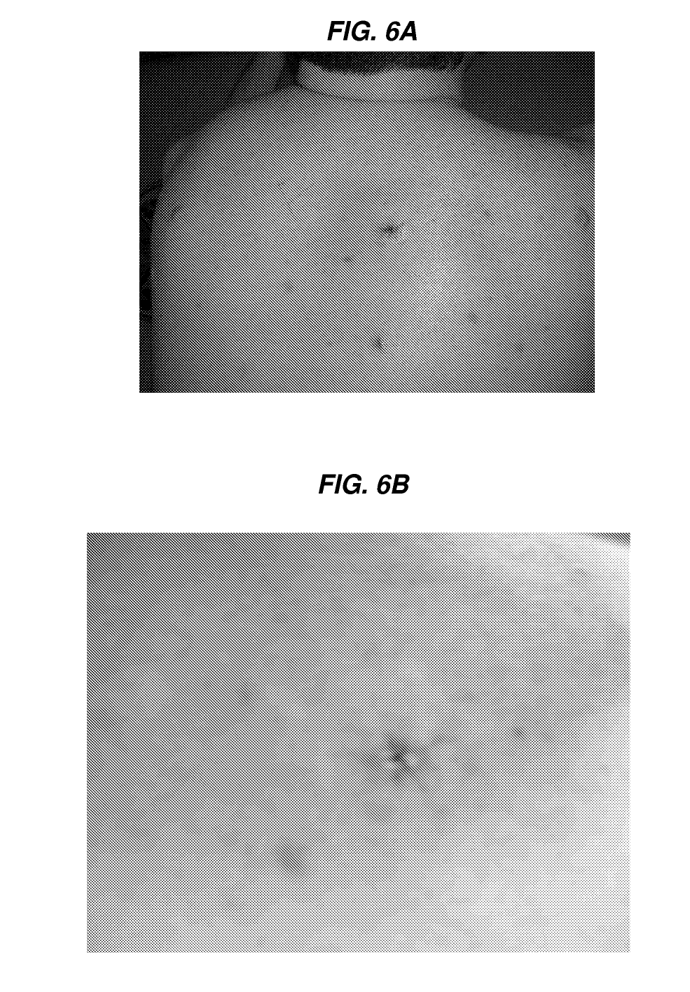

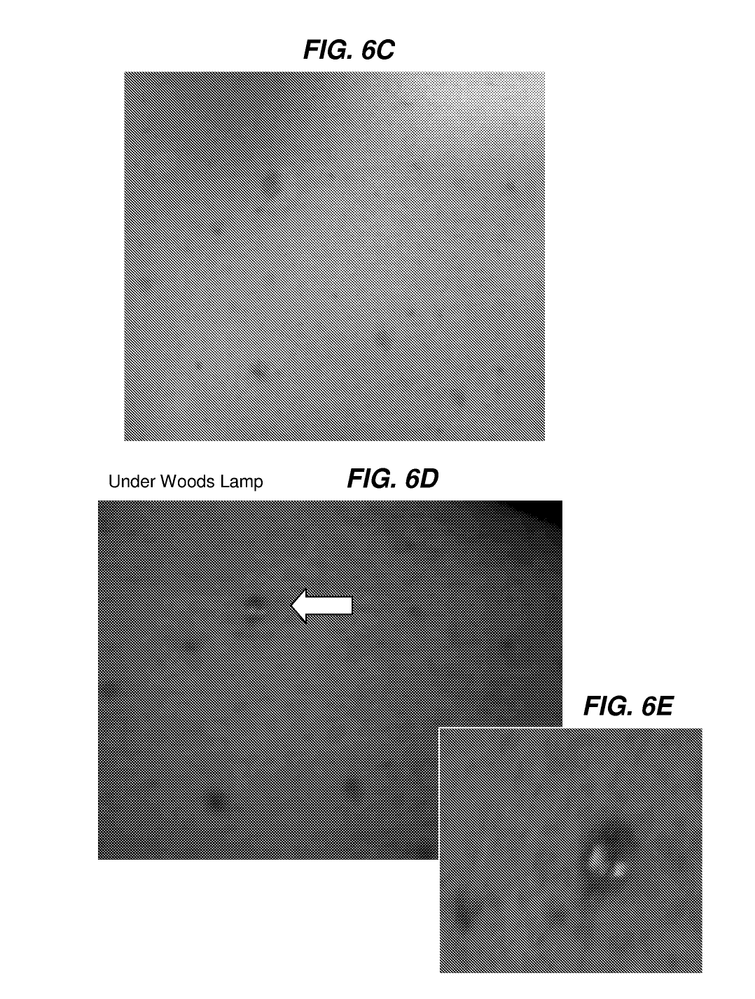

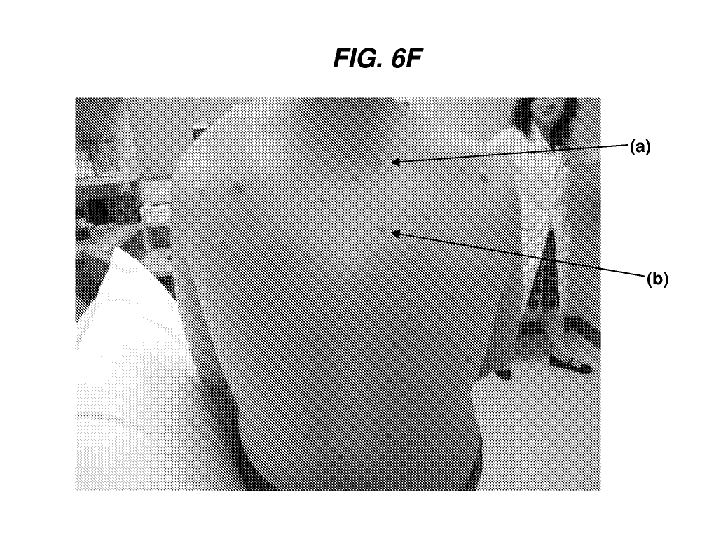

As shown in FIGS. 1A-1D and 6C-6F, a biocompatible dye, e.g., a fluorescent dye tattoo according to an embodiment of the invention is invisible under normal lighting conditions while under blacklight, as shown in FIGS. 1D-1E and 6D, the highlighted portions of the skin clearly show the pattern of the tattoo.

In some embodiments, a fluorescent pigment/vehicle complex comprising a biocompatible dye, e.g., a fluorescent dye and the vehicle, can be from about 1 to 700 microns in diameter, and more preferably from about 5 to about 300 microns in diameter. Particles of this size produce clear tattoos with little or no diffusion of the pigment to cause blurring of the lines. The size of the fluorescent pigment/vehicle complex is not of functional significance if an anchoring system is used to prevent spontaneous elimination or diffusion. That is, any size fluorescent pigment/vehicle complex is useable if the vehicle resists spontaneous elimination secondary to chemical bonding to the surrounding tissue, or if the complex is entrapped within cells.

The various possible morphologies of the fluorescent pigment/vehicle complexes include, but are not limited to, microspheres, microcapsules), microflakes, microparticles, liposomes, and coated pigment particles. The specific geometry of a fluorescent pigment/vehicle complex influences the amount of pigment or fluorescent dye required to produce the desired effect. In some embodiment, a thin-shelled microcapsules have a small percentage of polymer, generally about 0.5% or more, while solid microspheres or flakes may have a much higher percentage of polymer, in excess of 80% or more. The amount of fluorescent pigment or dye, as well as the morphology of the vehicle, can be varied depending upon the color of the fluorescent pigment or dye and the color of the skin on which the tattoo ink is to be used. One skilled in the art of preparing tattoo inks can readily determine without undue experimentation how much pigment or dye is required for each type of pigment/vehicle complex to produce the desired effect when administered into the dermis.

In some embodiments when the vehicle is in the form of microspheres, the microspheres can be either solid or hollow. The microspheres contain the fluorescent pigment or dye either throughout the substance of the vehicle, only in the internal portion of the vehicle, or only in the external portion of the vehicle. If a fluorescent pigment or dye is contained only in the internal portion of the vehicle, the overlying portion must be sufficiently translucent or transparent to permit the fluorescent pigment or dye color to be visible under the specific light excitations, e.g., UV or infared light. In some embodiments, microspheres possess specific characteristics, primarily size and immunoprotection, which resist and prevent spontaneous elimination from the dermis.

In some embodiments, microcapsules are microspheres with an outer shell and a central cavity or core. The outer shell of the microcapsule can be composed of a selected material with the desired stability characteristics, while the central cavity or core contains the fluorescent pigment or dye. In some embodiments, when the microcapsules are used to produce a permanent tattoo ink, the central cavity can contain the carrier and the outer shell can comprise the pigment or dye.

In some embodiments, microcapsules comprising the fluorescent dye can be constructed using methods known to those skilled in the art. For example, spheres can be formed by interfacial polymerization, hot melt microencapsulation, rotating cylinders or disks, solvent removal, solvent evaporation, or other methods known to those skilled in the art, including those disclosed in U.S. Pat. No. 4,898,734 to Mathiowitz et al. and No. 5,254,428 to Ishjikawa et al., which are incorporated in their entirety by reference.

For example, polyamide microcapsules can be constructed by interfacial polymerization using the method of Mathiowitz et al. in J. App. Poly. Sci., 26:809 (1981). In this method, an aqueous solution of the amine and polyvinyl alcohol along with the pigment to be encapsulated are added to a suspension of a benzene:xylene solution (2:1, v/v) of the dichloride in water. Azobisisobutyronitrile and/or azobenzene are added to the organic solution. The polycondensation reaction is allowed to continue for a desired period of time. Microcapsules are separated by decantation, repeatedly washed with distilled water, and dried by rapid washing with acetone.

In some embodiments, a fluorescent pigment/vehicle complex can also be produced in the form of microflakes which are small flat flakes of a selected material with the desired stability and physical characteristics. A fluorescent pigment or dye is mixed throughout the substance of the microflakes. This fluorescent pigment/vehicle complex morphology yields a larger surface to volume ratio as compared to microspheres, microcapsules, or microparticles. As the vehicle degrades, dissolves, absorbs, or erodes, the gross appearance of the flakes is relatively unaffected. Once a high percentage of the original material has eroded, only then does the gross appearance fade noticeably.

In some embodiments, a fluorescent pigment/vehicle complex can also be produced as coated particles. For example, a selected fluorescent pigment or dye is coated using any conventional technique with a material which encases the pigment, yielding a fluorescent particle (e.g., fluorescent pigment/vehicle complex) with characteristics which prevent spontaneous elimination from the dermis. If an erasable fluorescent tattoo is desired at the site of biopsy, then in some embodiments the coating material is one which is altered when a specific energy is applied, allowing the coating material to disrupt, allowing the pigment to be spontaneously eliminated. Alternatively, in some embodiments, if a semi-permanent fluorescent tattoo is desired at the biopsy location, a coating material can be one which bioabsorbs, bioerodes, dissolves, or biodegrades over a period of time, releasing the fluorescent pigment or dye for its eventual elimination.

In some embodiments, the tattooing fluorescent pigments or dyes can also be encapsulated in liposomes, such as those described in U.S. Pat. No. 4,900,556 to Wheatly et al., which is incorporated herein in its entirety by reference. Liposomes are highly advanced assemblages consisting of concentric closed membranes formed by water-insoluble polar lipids, particularly phospholipids. Other substances, such as cholesterol, can be included in the membrane. The stability, rigidity, and permeability of the liposomes are altered by changes in the phospholipid composition. Membrane fluidity is generally controlled by the composition of the fatty acyl chains of the lipid molecules. The fatty acyl chains can exist in an ordered, rigid state or in a relatively disordered fluid state. Factors affecting rigidity include chain length, degree of saturation of the fatty acyl chains and temperature. Larger chains interact more strongly with each other, so fluidity is greater with shorter chains. Saturated chains are more flexible than unsaturated chains. Transition of the membrane from the rigid to the fluid state occurs as the temperature is raised above the “melting temperature.” The melting temperature is a function of the length and degree of unsaturation of the fatty acyl chain.

Additionally, inclusion of a sterol, such as cholesterol, or a charged amphiphile, can alter the stability, rigidity, and permeability of the liposome by altering the charge on the surface of the liposome and increasing the distance between the lipid bilayers. Proteins and carbohydrates may be incorporated into the liposomes to further modify their properties.

Liposomes are conventionally prepared by dissolving an appropriate concentration of phospholipid in an organic solvent, evaporating the solvent, and subsequently disrupting the dry lipid layer with excess water or buffer. The fluorescent pigments or dyes can be entrapped within the liposomes during formation. “Entrapment” means the incorporation of the pigment or dye in the lipid framework of the bilayer or the passive encapsulation of the pigment or dye in the aqueous compartments.

In some embodiments, the liposomes can be designed to degrade upon exposure to a particular stimulus, such as light, heat, or sonic energy. In some embodiments, liposomes which undergo dramatic increases in permeability when irradiated with light are also known in the art. Examples of these photosensitive phospholipids include, but are not limited to, 1,2-diretinoyl-Sn-glycero-3-phosphocholine and 1-palmitoyl,2-retinoyl-Sn-glycero-3-phosphocholine. The permeability of liposomes formed from either or both of these phospholipids is directly proportional to temperature. Upon exposure to 30 to 120 seconds of 360 nm light, the permeability of the liposomes increases dramatically, from approximately 20% to almost 90%. Thus, fluorescent pigments or dyes encapsulated within such liposomes can be administered at the site of the biopsy, e.g., at the dermis for a cutaneous biopsy to produce a relatively permanent tattoo. When the owner of the tattoo wishes to erase the tattoo, or the physician wishes to remove the tattoo prior to, or during a subsequent biopsy procedure, the physician or the owner merely exposes the tattoo to from about 30 to 120 seconds of light at about 360 nm, and the liposomes become permeable, releasing the dye or pigment into the body from which the dye or pigment is slowly eliminated.

In some embodiments, a vehicle material for use with the fluorescent dye can be any biocompatible material that possesses the in vivo characteristics required for the type of fluorescent tattoo to be created at the site of the biopsy. For example, if a permanent fluorescent tattoo is desired at the site of the biopsy, the vehicle material is substantially inert and resists elimination, remaining indefinitely in the dermis. Alternatively, where it is desirable for an erasable fluorescent tattoo at the site of the biopsy, a vehicle material must be capable of releasing the pigment on demand upon imposition of a specific exogenous energy. Alternatively, in embodiments where a semi-permanent fluorescent tattoo is desirable at the location of the biopsy, the vehicle material must be bioabsorbable, bioerodable, or biodegradable over a predetermined period of time.

Among other materials that can function as fluorescent pigment vehicles for use in the methods, systems and devices as disclosed herein, which the FDA acceptable vehicles which have been approved for use as food additives, including succinylated gelatin, arabinogalactan, glutaraldehyde, petroleum wax, and mixtures thereof. Additional materials for use as pigment vehicles, according to the present invention, include poloxanele, poly(acrylic acid co-hypophosphorite) sodium salt, polyacrylamide, alginate/alginic acid, calcium caseinate, calcium polypectate, cellulose acetate phthalate, cellulose acetate trimellitate, chitosan, edible and natural waxes, fatty acids, fatty alcohols, gellan gums, hydroxy cellulose, hydroxy ethyl cellulose, hydroxy methyl cellulose, hydroxy propyl cellulose, hydro propyl ethyl cellulose, hydroxy propyl methyl cellulose phthalate, lipids, mono-, di- and triglycerides, pectins, phospholipids, polyalkyl(C.sub.16-C.sub.22)acrylate, polyethylene, oxidized polyethylene, polyethyleneimine reacted with 1,2-dichloroethane, polyoxyethylene(600)dioleate, polyoxyethylene(600)monoricinoleate, polyoxyethylene(23)lauryl ether, polyethylene glycol, polyethylene glycol(400)dioleate, polyethylene glycol(400)mono-& di-oleate, polyglycerol esters of fatty acids, polyisobutylene, polyglycerol phthalate ester of coconut oil fatty acids, polymaleic acid and/or its sodium salts, polyoxyethylene glycol(400)mono-& di-oleates, polyoxyethylene(23)lauryl ether, polyoxyethylene(40)monostearate, polyoxyethylene-poyoxypropylene block polymers, polyoxyethylene (20)sorbitan monooleate, polyoxyethylene(20)sorbitan monostearate, polyoxyethylene(2)sorbitan tristearate, polyoxypropylene glycol, polyvinyl acetate, polysorbate 80, polyvinylpolypyrrolidone, polyvinylpyrrolidone, and poly(20 vinylpyridine-co-styrene).

In some embodiments, other materials for forming the fluorescent pigment vehicles are biologically tolerated, and include but are not limited to waxes, polyolefins, or paraffins (e.g., Bayberry, spermaceti, Japan, Ross, etc.), triglycerides, phospholipids, fatty acids and esters thereof (e.g., lauric acid, palmitic acid, sorbitan monopalmitate, sorbitan monostearate, etc.), poly(vinyl palmitate), poly(hexadecyl acrylamide), poly(butyl acrylate), poly(hexadecyl acrylate), poly(octadecyl acrylate), poly(dodecene), poly(isobutene), poly(trimethyl glutarate), polyanhydides, polyorthoesters, polystyrene, polyurethane, polypropylene, polymethacrylate, polytetrafluoroethylene, and other known polymers, ceramics, or glasses.

The amount of fluorescent pigment or fluorescent dye used with a vehicle depends upon the desired color and intensity of the fluorescent pigment or dye, as well as the color and texture of the skin to which the fluorescent pigment or dye is to be administered. To form appropriate tattooing fluorescent ink for coating the cutting surfaces of the biopsy devices as disclosed herein, the fluorescent pigment/vehicle complexes are formed into microstructures of desired composition and geometry and then suspended in a physiologically acceptable carrier, such as ethanol or water, or any other conventional acceptable carrier, in a concentration sufficient to produce the desired fluorescent pigmentation at the site of the biopsy, e.g., at the surface of the skin where the tissue was removed.

Alternatively, in some embodiments, a fluorescent pigment/vehicle complex can be in the form of a suspension in a semi-liquid paste which can be easily used to coat the cutting surface of the biopsy device. In such embodiments, the size of the fluorescent pigment/vehicle complex is selected so that the fluorescent dye easily coats and adheres to the cutting surface of the biopsy device, yet is administered into the surface of the tissue during the biopsy procedure, e.g., into the dermis.

In some embodiments, suitable fluorescent or phosphorescent pigments or dyes are used and incorporated a pigment vehicle as desired and disclosed herein. In some embodiments, a composition of the vehicle is selected according to whether the biopsy marking fluorescent tattoo is to be permanent, semi-permanent , or erasable. In embodiments where it is desirable to have a biopsy marking fluorescent tattoo semi-permanent, a pigment vehicle is chosen which bioabsorbs, bioerodes, or biodegrades at the predetermined time the tattoo is to disappear spontaneously.

Any conventional fluorescent pigments or dyes suitable for tattoos can be used for the biopsy marking fluorescent tattoo inks of the present invention, as well as any biologically tolerated fluorescent and phosphorescent molecules. The Food and Drug Administration considers the pigments used in tattooing to be “color additives” subject to the FDA color additive regulations under the Federal Food, Drug and Cosmetic Act. [cf. 21 U.S.C. Sections 321(t) and 379(e)]. In addition, virtually any fluorescent pigment or substance tolerated by the body can be used as an appropriate biopsy marking fluorescent tattoo ink when incorporated with a pigment vehicle to form a fluorescent pigment/vehicle complex according to the present invention.

In some embodiments, a fluorescent pigment/vehicle complex is produced with the vehicle which comprises the pigment per se. Water soluble pigments (i.e., pigments that do not possess the necessary characteristics of remaining indefinitely in the dermis) are modified in a specific manner physically or chemically (i.e., aggregated, cross-linked) to provide the necessary characteristics to resist spontaneous elimination from the dermis. In essence, such modifications to the fluorescent pigment confer upon the fluorescent pigment itself the functional qualities of both fluorescent pigment vehicle and the fluorescent colorant. These fluorescent pigments are therefore modified to become their own tattoo pigment vehicle and, therefore, do not require a separate microstructure or composition to form a fluorescent pigment/vehicle complex. Furthermore, this alternative configuration can be made so that fluorescent pigment complex spontaneously disappears after a predetermined time period (semi-permanent tattoo) or is susceptible to a specific external energy, such as thermal, sonic (including ultrasonic, audible, and subsonic), light (including laser light), electric, magnetic, chemical, enzymatic, mechanical (such as shear force from rubbing or massaging), or any other type of energy or combination of energies. Treatment of the biopsy marked fluorescent tattooed skin with the appropriate energy sufficiently alters the fluorescent tattoo pigment vehicle physically or chemically, allowing for elimination of the pigment and thus erasing the fluorescent tattoo marking the biopsy site on demand (erasable tattoo).

### Dye Formulations and Acceptable Carriers

In some embodiments, a biopsy site dye marker, e.g., a fluorescent dye having features of the present invention may be delivered to a biopsy site in dry form, or in wet form, as in a slurry or suspension. Pressure may be applied to the powder in order to eject it from a storage location, such as a delivery tube. Pressure effective to deliver a dye marker e.g., a fluorescent dye having features of the invention includes gas pressure, acoustic pressure, hydraulic pressure, and mechanical pressure.

In some embodiments, mechanical pressure may be delivered by, for example, direct contact with a plunger. In some embodiments, a method for delivering a dye marker e.g., a fluorescent dye to a biopsy site utilizes a biocompatible liquid to drive or carry the powder into the biopsy cavity at the biopsy site. For example, a quantity of a dye marker e.g., a fluorescent dye may be contained within a tube or chamber that leads directly or indirectly to a biopsy site. In some embodiments, the dye marker e.g., a fluorescent dye may be dispensed by the application of hydraulic pressure applied by a syringe containing sterile saline or other suitable liquid.

In some embodiments, a dye marker e.g., a fluorescent dye marker is contained within a tube termed a “delivery tube” or “delivery device”. The tube has an outside diameter that is sized to fit within a cannula which can be attached to the biopsy device, the exact dimensions of the tube will depend on the biopsy device used. In addition, a delivery tube may have markings to aid in determining the depth of the tube within a cannula, surface features (such as pins, slots, bumps, bars, wedges, luer-lock fittings, or bands, including a substantially conical circumferential band) effective to control the depth into which a delivery tube is fitted within a cannula or effective to lock a delivery tube into position within a cannula. For example, a delivery tube may have pins or bumps configured to engage a slot or a leading edge of a cannula, or a luer-lock fitting configured to lock into a cannula.

A biopsy device as disclosed herein may also be configured to receive and to engage a delivery tube. A biopsy device as disclosed herein may have pins, slots, wedges, bumps, bands, luer-lock fittings, or the like, to engage a delivery tube and to hold it into a desired position within the biopsy device. For example, a biopsy device as disclosed herein may have a luer-lock fitting, or a slot to engage a pin on a delivery tube, or an internal bump wedge or band that limits the distance of travel of the delivery tube within the biopsy device. Delivery tubes embodying features of the present invention may be made of any suitable bio-compatible material.

A dye marker e.g., a fluorescent dye which is a fluid can also be used to deposit the dye marker at a biopsy site. In some embodiments, the dye marker e.g., a fluorescent dye may contain other agents, including inert agents, osmotically active agents, pharmaceutical agents, and other bio-active agents. For example, a suitable biocompatible liquid may be selected from the group consisting of sterile saline, sterile saline containing a pharmaceutical agent, sterile saline containing an anesthetic agent, sterile saline containing a hemostatic agent, sterile saline containing a colorant, sterile saline containing a radio contrast agent, sterile sugar solution, sterile sugar solution containing a pharmaceutical agent, sterile sugar solution containing an anesthetic agent, sterile sugar solution containing a hemostatic agent, sterile sugar solution containing a colorant, sterile sugar solution containing a radio contrast agent, biocompatible oils, biocompatible oils containing a pharmaceutical agent, biocompatible oils containing an anesthetic agent, biocompatible oils containing a hemostatic agent, biocompatible oils containing a radio contrast agent, and biocompatible oils containing a colorant. For example, anesthetic agents may be beneficial by reducing patient discomfort.

In some embodiments, a formulation of a dye marker e.g., a fluorescent dye can optionally comprise hemostatic agents to help reduce bleeding, enhance clotting, or to cause vasoconstriction in a patient. Hemostatic agents include adrenochrome, algin, alginic acid, aminocaproic acid, batroxobin, carbazochrome salicylate, cephalins, cotarmine, ellagic acid, epinephrine, ethamsylate, factor VIII, factor IX, factor XIII, fibrin, fibrinogen, naphthoquinone, oxamarin, oxidized cellulose, styptic collodion, sulamrin, thrombin, thromboplastin (factor III), tolonium chloride, tranexamic acid, and vasopression.

In some embodiments, a formulation of a dye marker e.g., a fluorescent dye can optionally comprise at least one pharmaceutical agent, for example, to promote healing, and to treat injury, infection, and diseases such as cancer, and may include, for example, but is not limited to hormones, hemostatic agents and anesthetics as well as antibacterial, antiviral, antifungal, anticancer, and other medicinal agents. Accordingly, in some embodiments, pharmaceutical agents may be included as part of a formulation of a dye marker e.g., a fluorescent dye placed within a biopsy cavity in order, for example, to promote healing, prevent infection, and to help treat any cancer cells remaining near the biopsy site.

In some embodiments it may be desirable to add bioactive molecules to a formulation comprising a dye marker e.g., a fluorescent dye. A variety of bioactive molecules can be delivered using the matrices described herein. These are referred to generically herein as “factors” or “bioactive factors”. In some embodiments, a bioactive factor is selected from any of the following: growth factors, angiogenic factors, compounds selectively inhibiting in-growth of fibroblast tissue such as anti-inflammatory agents, and compounds selectively inhibiting growth and proliferation of transformed (cancerous) cells. These factors may be utilized to control the growth and function of implanted cells, the in-growth of blood vessels into the forming tissue, and/or the deposition and organization of fibrous tissue around the implant.

Examples of growth factors include heparin binding growth factor (HBGF), transforming growth factor alpha or beta (TGF-beta), alpha fibroblastic growth factor (FGF), epidermal growth factor (TGF), vascular endothelium growth factor (VEGF), some of which are also angiogenic factors. Other factors include hormones such as insulin, glucagon, and estrogen. In some embodiments it may be desirable to incorporate factors such as nerve growth factor (NGF) or muscle morphogenic factor (MMP).

In some embodiments, growth factors also include factors which promote skin healing and reduce scarring. For example, pharmaceutical agents such as hemostatic, analgesic, or anesthetic agents may also be incorporated into formulation of a dye marker e.g., a fluorescent dye coated on a cutting surface of the biopsy device. Hemostasis-promoting agents help to prevent the formation of hematomas and can also help to promote the healing process. In some embodiments, a formulation of a dye marker e.g., a fluorescent dye can also comprise an agent which emits a therapeutic radiation to treat any cancerous tissue remaining in the margin of the biopsy cavity.

In some embodiments, steroidal anti-inflammatory agent can be used to decrease inflammation to the implanted dye marker e.g., a fluorescent dye, thereby decreasing the amount of fibroblast tissue growing at the site of the biopsy. Such factors are well known to those skilled in the art and are available commercially or described in the literature. In some embodiments, bioactive factors which are optionally combined with a formulation comprising a dye marker e.g., a fluorescent dye are incorporated to between one and 30% by weight, although the factors can be incorporated to a weight percentage between 0.01 and 95 weight percentage.

Bioactive molecules can be incorporated into a formulation comprising a dye marker e.g., a fluorescent dye and can be released over time by diffusion and/or degradation of the dye marker, they can be incorporated into microspheres which are attached to or incorporated within the marker, or some combination thereof.

### Definitions

The term “fluorescent” refers to the luminescence, or emission of visible light that occurs where the energy of a specific wavelength is supplied by electromagnetic radiation, usually ultraviolet light. The energy source kicks an electron of an atom from a lower energy state into an “excited” higher energy state; then the electron releases the energy in the form of light (luminescence) when it falls back to a lower energy state. Fluorescent dye as disclosed herein refers to a dye which exhibits energy and is visible under a illumination at a predefined wavelength. Numerous fluorescent molecules are commercially available and can be adapted for use in the methods, systems, devices and kits as disclosed herein, and include those from molecular probes, Sigma and similar other commercial sources.

The term “phosphorescence” refers to the emission of visible light from a molecule containing phosphorus that has been exposed to a radiation source, e.g., electromagnetic radiation which continues beyond a few nanoseconds after radiation has ceased.

The term “tattoo” as used herein refers to permanent or semi-permanent mark made on the surface of a tissue, for example a skin tissue by a process of inserting a pigment or dye into the surface of the tissue.

The term “biopsy” as used herein refers to the removal of tissue cells and/or a tissue mass from the living body, typically for examination for diagnosis of a disease or disorder. The term biopsy can refer to a skin biopsy. Different methods of biopsy procedures are encompassed in the invention, and include without limitation aspiration biopsy (biopsy in which tissue is obtained by application of suction through a needle attached to a syringe), brush biopsy (biopsy in which cells or tissue are obtained by manipulating tiny brushes against the tissue or lesion in question (e.g., through a bronchoscope) at the desired site), cone biopsy biopsy in which an inverted cone of tissue is excised, as from the uterine cervix, core biopsy (core needle biopsy needle biopsy with a large hollow needle that extracts a core of tissue), endoscopic biopsy (removal of tissue by appropriate instruments through an endoscope), excisional biopsy (biopsy of tissue removed by surgical cutting), incisional biopsy (biopsy of a selected portion of a lesion), needle biopsy, also percutaneous biopsy (biopsy in which tissue is obtained by puncture of a tumor, the tissue within the lumen of the needle being detached by rotation, and the needle withdrawn), punch biopsy (biopsy in which tissue is obtained by a punch), shave biopsy (biopsy of a skin lesion in which the sample is excised using a cut parallel to the surface of the surrounding skin), stereotactic biopsy (biopsy of the brain using stereotactic surgery to locate the biopsy site), sternal biopsy (biopsy of bone marrow of the sternum removed by puncture or trephining).Other biopsys included in the term biopsy include; Abdominal wall fat pad biopsy, Agonal biopsy, Aspiration biopsy, Biochemical biopsy, Blastocyst biopsy, Blind biopsy, Bone marrow aspiration & biopsy, Breast biopsy, Cervical biopsy, Chorionic villus biopsy, Cleavage stage biopsy, Cold cone biopsy, Cone biopsy, Core biopsy, Endobronchial biopsy, Endometrial biopsy, Endomyocardial biopsy, Endoscopic biopsy, Excisional biopsy, Fine needle aspiration biopsy, Guided wire open biopsy, Heart biopsy, Incisional biopsy, Jumbo biopsy, Metabolic biopsy, Microbiopsy, Mirror image biopsy, Muscle biopsy, Needle biopsy, Nerve biopsy, Open biopsy, Open lung biopsy, Pleural biopsy, Polar body biopsy, Prostate biopsy, Punch biopsy, Renal biopsy, Salivary gland biopsy, Saucerization biopsy, Sentinel lymph node biopsy, Sextant biopsy, Shave biopsy, Skin biopsy, Skinny biopsy, Skinny needle biopsy, Small intestinal biopsy, Stereotactic biopsy, Stereotactic needle biopsy, Transbronchial needle biopsy, Transbronchial biopsy, Wedge biopsy, Wire-guide excisional biopsy. Other biopsy procedures include aspiration biopsy (biopsy in which tissue is obtained by application of suction through a needle attached to a syringe), bite biopsy (instrumental removal of a fragment of tissue), closed biopsy (one carried out without access through an open incision such as a laparotomy. An example is a percutaneous, fine needle aspirate), cone biopsy (biopsy in which an inverted cone of tissue is excised), biopsy dart (a dart which cuts a skin bipsy, then falls out, which is useful for superficial lesions), endoscopic biopsy (removal of tissue by appropriate instruments through an endoscope), excisional biopsy (biopsy of tissue removed from the body by surgical cutting), exploratory biopsy (a combination of exploratory surgery to determine size and location of a lesion and the taking of a biopsy), fine needle biopsy and needle biopsy (biopsy in which tissue is obtained by puncture of a tumor, the tissue within the lumen of the needle being detached by rotation, and the needle withdrawn), incisional biopsy (biopsy of a selected portion of a lesion), needle biopsy, punch biopsy, Robson-Heggers biopsy (a procedure for the collection of a piece of tissue from an infected wound in order to determine the extent and the nature of the infection), surface biopsy (sample of cells scraped from the surface of a lesion or obtained by impression smears), surgical biopsy (one obtained during a surgical procedure), synovial biopsy (by a needle biopsy technique or through an arthrotomy incision using special forceps for a bite biopsy), total biopsy (obtained by removal of the entire lesion, for example for both therapeutic as well as diagnostic purposes), ultrasound-guided biopsy (use of ultrasonography to guide the passage of a needle or biopsy instrument into a lesion).

The term “biopsy device” refers to any apparatus which can be used to conduct a biopsy procedure. In some embodiments, the biopsy device is a disposable biopsy device. In alternative embodiments, the biopsy device comprises a disposable portion and a non-disposable portion, usually a handle, where the disposable portion comprises a tissue cutting surface, such as a blade such as a scalpel blade, e.g., a microscapel blade, or punch biopsy core cutting edge etc. In some embodiments, the biopsy device is metallic, and in some embodiments, the biopsy device is a non-metallic composition, e.g., diamond, glass and other hard substances suitable for cutting tissue of a subject. In some embodiments, the biopsy device comprises a laser cutting technology.

The term “tissue cutting edge” refers to a surface, usually sharp edge of the biopsy device which when it contacts with the surface of a tissue, e.g., skin, makes an incision in the tissue to excise a part of the tissue from the subject, so the tissue can be excised and removed from the subject as the biopsy tissue sample. In some embodiments, pressure is needed for the tissue cutting edge to make an incision in the tissue when it contact the surface of the tissue. In some embodiments, the cutting edge makes a penetrating incision and in some embodiments the tissue cutting edge makes a slice incision. A tissue cutting edge can be used for press cutting of the surface of the tissue, e.g., the surface of the skin, where press cutting is a technique for making an incision with a tissue cutting edge, e.g., a scalpel blade, in which there is increasing pressure in the same direction the blade is being moved. A tissue cutting edge can be used for slide cutting of the surface of the tissue, e.g., the surface of the skin, where pressure is applied at right angles, e.g., at a tangent to the movement of the blade. A tissue cutting edge which can be used for slide cutting can often be better controlled than press cutting when performing a biopsy procedure.

The term “biopsy cavity” refers to the void in the subject's tissue after a tissue mass is removed (e.g., a biopsy tissue sample) is removed after a biopsy procedure.

The term “biopsy cavity wall” refers to the tissue boundary where the tissue mass has been removed from the subject after the biopsy procedure. Stated another way, the biopsy cavity wall refers to the surface of the tissue, where the surface is an external tissue surface (e.g, the wall) a void which is created when a tissue mass was removed from the tissue.

The term “pigment” or “dye” refers to molecules, e.g., fluorescent molecules which are capable of being visualized under a pre-defined wavelength.

The term “carrier” as used herein means an acceptable material, composition or vehicle, such as a liquid or solid filler, diluent, excipient, solvent or encapsulating material, involved in suspending the biocompatible dye, e.g., to be deposited on a tissue cutting edge of the biocompatible device, or to be deposited in the subject. Each carrier must be “acceptable” in the sense of being compatible with the other ingredients of the formulation. Thus, in some embodiments, the carrier is a pharmaceutically acceptable carrier.

The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In some embodiments, the carrier comprises conventional additives depending on administration form for example, in one embodiment the carrier for the biocompatible dye is in a form suitable for injections. Conventional carrier substances, such as isotonic saline, may be used.

The term “vehicle” refers to encapsulating material or an inert substance combined with, or surrounding the biocompatible dye to facilitate administration or application. In some embodiments, the vehicle serves as a solvent, or in alternative embodiments to increase the bulk of the dye.

The term “biocompatible” as used herein refers to a substance or agent, e.g., a dye molecule which is compatible with living cells, tissues, organs, or systems, and posing no or little risk of injury, toxicity, or rejection by the immune system.

The term “pathology” as used herein, refers to symptoms, for example, structural and functional changes in a cell, tissue, or organs, which contribute to a disease or disorder. For example, the pathology may be associated with a particular nucleic acid sequence, or “pathological nucleic acid” which refers to a nucleic acid sequence that contributes, wholly or in part to the pathology, as an example, the pathological nucleic acid may be a nucleic acid sequence encoding a gene with a particular pathology causing or pathology-associated mutation or polymorphism. The pathology may be associated with the expression of a pathological protein or pathological polypeptide that contributes, wholly or in part to the pathology associated with a particular disease or disorder. In another embodiment, the pathology is for example, is associated with other factors, for example ischemia and the like.

The term “biological sample” as used herein refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, whole blood, plasma, serum, urine, semen, saliva, aspirates, cell culture, or cerebrospinal fluid. Biological samples also include tissue biopsies, cell culture. A biological sample or tissue sample can refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tissue biopsies, scrapes (e.g. buccal scrapes), tumors, organs, and also samples of in vitro cell culture constituent. In some embodiments, where the sample is solid, it can be liquidized and homogenized into a liquid sample for use in the device and systems as disclosed herein. In some embodiments, the sample is from a resection, bronchoscopic biopsy, or core needle biopsy of a primary or metastatic tumor, or a cellblock from pleural fluid. In addition, fine needle aspirate samples are used. Samples may be either paraffin-embedded or frozen tissue. The sample can be obtained by removing a sample of cells from a subject, but can also be accomplished by using previously isolated cells (e.g. isolated by another person), or by performing the methods of the invention in vivo. Biological sample also refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the biological samples can be prepared, for example biological samples may be fresh, fixed, frozen, or embedded in paraffin.

The term “tissue” is intended to include intact cells, blood, blood preparations such as plasma and serum, bones, joints, muscles, smooth muscles, and organs.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, interdisposition, affection. A disease and disorder, includes but is not limited to any condition manifested as one or more physical and/or psychological symptoms for which treatment is desirable, and includes previously and newly identified diseases and other disorders.

The articles “a” and “an” are used herein to refer to one or to more than one (i.e., to at least one) of the grammatical object of the article. By way of example, “an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean ±5%. The present invention is further explained in detail by the following examples, but the scope of the present invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Although this invention has been described in terms of certain preferred embodiments, other embodiments which will be apparent to those of ordinary skill in the art in view of the disclosure herein are also within the scope of this invention. Accordingly, the scope of the invention is intended to be defined only by reference to the appended claims. All documents cited herein are incorporated herein by reference in their entirety.

In some embodiments of the present invention may be defined in any of the following numbered paragraphs:

1. A tissue marking system comprising combining a biopsy device with a biocompatible dye, wherein the biopsy device applies the biocompatible dye to the biopsy cavity walls or surface of a subject's tissue during a biopsy procedure, and wherein the biocompatible dye is visible under a predetermined wavelength.

2. The tissue marking system of claim 1, wherein the biocompatible dye is a fluorescent dye.

3. The tissue marking system of claim 1, wherein the biocompatible dye is not visible or minimally visible under normal (white) light.

4. The tissue marking system of claim 1, wherein the biocompatible dye is visible under UV light or black light

5. The tissue marking system of claim 1, wherein the biocompatible dye is visible at under 400 nm wavelength.

6. The tissue marking system of claim 1, wherein the biocompatible dye is visible at about 365 nm wavelength.

7. The tissue marking system of claim 1, wherein the device comprises a tissue cutting edge.

8. The tissue marking system of claim 7, wherein the cutting edge is coated with the biocompatible dye.

9. The tissue marking system of claim 1, wherein the tissue is skin.

10. The tissue marking system of claim 1, wherein the tissue is the dermis.

11. The tissue marking system of any of claims 1 to 10, wherein the biopsy device is a cutaneous biopsy device.

12. The tissue marking system of any of claims 1 to 11, wherein the biocompatible dye is semi-permanent.

13. The tissue marking system of any of claims 1 to 12, wherein the biocompatible dye degrades after a predetermined time.

14. The tissue marking system of any of claims 1 to 13, wherein the biocompatible dye degrades after at least 6 months.

15. The tissue marking system of any of claims 1 to 13, wherein the biocompatible dye degrades after at least 12 months.

16. The tissue marking system of any of claims 1 to 13, wherein the biocompatible dye degrades after at least 2 years.

17. A biopsy device comprising at least one tissue cutting edge, wherein at least one tissue cutting edge is coated with a biocompatible dye, wherein the biocompatible dye is reactive under a predefined wavelength.

18. The biopsy device of claim 17, wherein the biocompatible dye is a fluorescent dye.

19. The biopsy device of claim 17, wherein the biocompatible dye is not visible or minimally visible under normal (white) light.

20. The biopsy device of claim 17, wherein the biocompatible dye is visible under UV light or black light.

21. The biopsy device of claim 17, wherein the biocompatible dye is visible at under 400 nm wavelength.

22. The biopsy device of claim 17, wherein the biocompatible dye is visible at about 365 nm wavelength.

23. The biopsy device of claim 17, wherein the biocompatible dye is visible at about 365 nm wavelength.

24. The biopsy device of claim 17, wherein the biopsy device is a cutaneous biopsy device.

25. The biopsy device of claim 24, wherein the cutaneous biopsy device is a cutaneous needle biopsy device.

26. The biopsy device of claim 17, wherein the biopsy device is selected from the group consisting of: needle biopsy device, hookwire biopsy device, photonic needle, clamp, forceps, micro-scissors, punch biopsy device, core biopsy device, razor, scapel blade, suture, shave biopsy device, a cutaneous needle biopsy device.

27. The biopsy device of claim 17, wherein the biopsy device is used for a punch biopsy.

28. The biopsy device of claim 17, wherein the biopsy device is used for a shave biopsy.

29. The biopsy device of claim 17, wherein the biopsy device is a disposable biopsy device.

30. The biopsy device of claim 29, wherein the disposable biopsy device is a scalpel blade.

31. The biopsy device of claim 29, wherein the disposable biopsy device is a flexible blade.

32. The biopsy device of claim 29, wherein the disposable biopsy device is a suture.

33. A method of determining the site of a biopsy, comprising:
a. using the system of any of claims 1 to 11 or the biopsy device of any of claims 12 to 27 when an initial biopsy is being performed to mark the site of the biopsy;
b. locating the biopsy site at a subsequent timepoint by illuminating the skin with the predefined wavelength.

34. A method of marking the site of a biopsy, comprising using the system of any of claims 1 to 11 or the biopsy device of any of claims 12 to 27 to mark the site where a biopsy is being performed, wherein the mark can be detected at subsequent timepoint by illuminating the site of the biopsy with the predefined wavelength.

35. A method for identifying the location of a biopsy site wherein the biopsy site was previously marked, comprising illuminating the skin with a predefined wavelength, and wherein the biopsy site was marked at the time of the biopsy procedure using the system of any of claims 1 to 11 or the biopsy device of any of claims 12 to 27 with a marker of the predetermined wavelength.

36. The method of any of claims 33 to 35, wherein the illuminating occurs at a timepoint that is after the marking of the site of the biopsy.

37. The method of claim 36, wherein the illuminating is at least 1 week after the marking of biopsy site.

38. The method of claim 37, wherein the illuminating is at least 2 weeks after the marking of biopsy site.

39. The method of claim 38, wherein the illuminating is at least 1 month after the marking of biopsy site.

40. The method of claim 39, wherein the illuminating is at least 2 months after the marking of biopsy site.

41. The method of claim 40, wherein the illuminating is at least 3 months after the marking of biopsy site.

42. The method of claim 41, wherein the illuminating is at least 6 months after the marking of biopsy site.

43. The method of claim 42, wherein the s illuminating is more than 6 months after the marking of biopsy site.

44. A method of marking a biopsy site comprising;
a. identifying a target area of a subjects skin for a biopsy procedure;
b. using the device of any of claims 12 to 27 comprising a biocompatible dye which is reactive at a predetermined wavelength, wherein the biopsy device is inserted into a tissue mass of the subject to be removed by the biopsy procedure; and
c. causing the biopsy device in the tissue mass to deposit the biocompatible dye in a biopsy cavity of the tissue mass as the tissue mass is removed by the biopsy device.

45. The method of claim 44, wherein biopsy device comprises a tissue cutting edge.

46. The method of claim 45, wherein the tissue cutting edge of the biopsy device is coated with the biocompatible dye, and wherein insertion of the tissue cutting edge of the biopsy device into the tissue mass deposits the biocompatible dye in a biopsy cavity of the tissue mass as the tissue mass is removed by the biopsy device.

47. A kit comprising a container comprising a biocompatible dye for coating a tissue cutting edge of a biopsy device.

48. The kit of claim 47, further comprising a biopsy device.

49. The kit of claim 48, wherein the biopsy device is a disposable biopsy device or a disposable attachment for a non-disposable biopsy device.

50. The kit of claim 49, wherein the disposable attachment for a non-disposable biopsy device comprises at least one tissue cutting edge.

51. The kit of any of claims 47 to 50, further comprising an apparatus to aid coating the tissue cutting edge of a biopsy device with the biocompatible dye.

52. The kit of any of claims 47 to 51, wherein the biocompatible dye is a fluorescent dye.

53. The kit of any of claims 47 to 52, wherein the biocompatible dye is not visible or minimally visible under normal (white) light.

54. The kit of any of claims 47 to 53, wherein the biocompatible dye is visible under UV light or black light.

55. The kit of any of claims 47 to 54, wherein the biocompatible dye is visible at under 400 nm wavelength.

56. The kit of any of claims 47 to 55, wherein the biocompatible dye is visible at about 365 nm wavelength.

57. The kit of any of claims 47 to 56, wherein the biocompatible dye is visible at less than about 365 nm wavelength.

58. A kit comprising at least one disposable biopsy device, wherein the disposable biopsy device has at least one tissue cutting edge, and wherein the at least one tissue cutting edge is coated with a biocompatible dye which is reactive under a predetermined wavelength.

59. The kit of claim 58, wherein the kit comprises at least 5 disposable biopsy devices.

60. The kit of claim 58, wherein the kit comprises at least 10 disposable biopsy devices.

61. The kit of claim 60, wherein the kit comprises at least 15 disposable biopsy devices.

62. The kit of claim 62, wherein the kit comprises at least 20 disposable biopsy devices. p 63. The kit of claim 62, wherein the kit comprises more than 20 disposable biopsy devices.

64. The kit of claim 58, wherein the disposable biopsy device is a disposable attachment comprising at least one tissue cutting edge which can be attached to a non-disposable biopsy device.

65. The kit of claim 64, wherein the disposable biopsy device comprises at least one tissue cutting edge selected from the group consisting of: scalpel blade, flexible blade, sutures and the like.

66. The kit of claim 64, wherein the disposable attachment comprising at least one tissue cutting edge is selected from the group consisting of: scalpel blade, flexible blade, sutures and the like.

67. Use of the biopsy device of any of claims 17 to 32 for marking the location of a biopsy site.

68. Use of the tissue marking system of any of claims 1 to 16 for determining the site of a biopsy in a subject.

### EXAMPLES

The examples presented herein relate to a biopsy device, methods, kits and systems for their use for marking a location of a biopsy site in order to later identify the location of a biopsy or surgery in a subject, where the marker is a dye, e.g., a fluorescent dye or tattoo which is not visible to the naked eye under normal (white) light, but is visible under a predetermined wavelength, e.g., UV light or blacklight. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Example 1

In vitro study demonstrating fluorescence tattoo is invisible under normal light and visible under UV light after multiple washes.

Methods:

Human fetal foreskin is harvested, and the biocompatible dye, e.g., fluorescent tattoo deposited to each sample using a biopsy device, e.g., needle biopsy device, and the human fetal foreskin tissue was washed in a combination of media and alcohol and cultured in Transwell microplate. Each day the human fetal foreskin tissue samples were removed under the ventilated hood and was washed with both media and alcohol (to mimics daily skin care). The human fetal foreskin tissue samples were harvested on Day 5 following application of the dye and analyzed using histological staining.

The inventors used neonatal foreskin as an example of human skin. The inventors assessed the following: (a) ease of application and the feasibility of applying the tattoo, (b) the optimal protocol for the application of the fluorescent tattoo, (c) the visibility of the tattoo fluorescence due under spectral light, e.g., normal (white) light and UV light or blacklight (e.g., Wood's lamp), (d) assessment of localization of the tattoo to the area of inoculation, and (e) visibility of the fluorescence tattoo under microscopy.

The inventors demonstrated that the biocompatible dye, e.g., a fluorescent dye tattoo can be applied or deposited on skin sample using a punch biopsy (e.g., core punch) device, in particular where the tissue cutting edges of the punch biopsy device is coated with the biocompatible dye, e.g., a fluorescent dye. The inventors determined that the biocompatible dye, e.g., a fluorescent dye localizes in area of inoculation and does not migrate from the site of deposit.

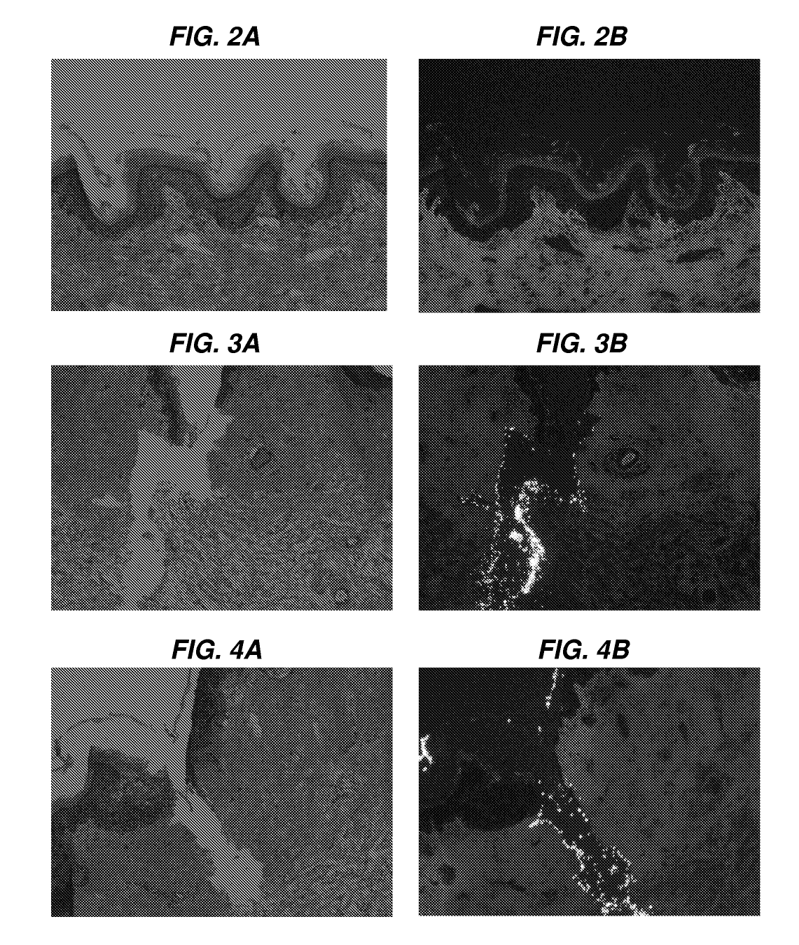

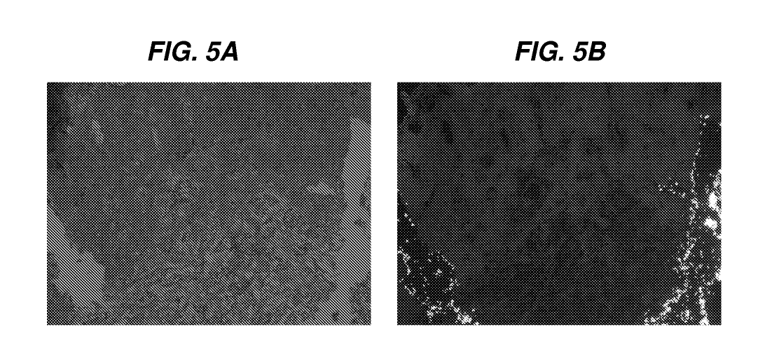

As shown in FIG. 1A, the inventors demonstrated that the deposited biocompatible dye, e.g., a fluorescent dye is invisible under normal (visible white) light and is visible under UV light (FIG. 1B) and wood's lamp (UV) (FIG. 1C and 1D). The inventors demonstrate that the biocompatible dye, e.g., a fluorescent dye is deposited on the biopsy cavity walls of the tissue sample as determined by histology staining (see FIGS. 2A-2B, 3A-3B, 4A-4B and 5A-5B). In particular, the biocompatible dye, e.g., a fluorescent dye is deposited from the tissue cutting surface of the biopsy device, such as a punch biopsy device, where the cutting edge is inserted into the tissue mass to be removed, thereby identifying the boundary of the biopsy site, where the tissue sample was removed from the remaining surrounding tissue.

Accordingly, the inventors have determined an easy system and method for deposited biocompatible dye, e.g., a fluorescent dye at the exact location of the biopsy site at the time of the biopsy procedure. The inventors also demonstrate use of a biopsy tool with the tissue cutting surfaces coated with a biocompatible dye, e.g., a fluorescent dye which easily and consistently can be used to localize a suspicious lesions on the subjects tissue, e.g. on the surface of the skin. Accordingly, the inventors have demonstrated that the depositing a biocompatible dye, e.g., a fluorescent dye at the location of a biopsy is an effective method, tool and system which can be safely incorporated into practitioners, e.g., physicians or dermatologists practice for performing a biopsy procedure of a lesions on a tissue surface, e.g., a subjects skin and then monitoring the biopsy site at subsequent follow-up appointments at one or more future dates.

The inventors have also demonstrated that the deposited biocompatible dye, e.g., a fluorescent dye can be permanent (data not shown), or in some instances, can be semi-permanent (data not shown). The inventors also demonstrated that no side effects were detected with the deposited biocompatible dye, e.g., a fluorescent dye in subjects for at least 6 months or more.

### Example 2

In vivo study demonstrating fluorescence tattoo is invisible under normal light and visible under UV light for at least 6 months following.

The inventors demonstrated the use of the biopsy device and the system and methods as disclosed herein on a subject (23 year male) with basal cell nervus syndrome with a lesion in the middle of his back, which was suspected to be a recurrent basal cell carcinoma (BCC) with multiple LN2 and ED&C by FP (see FIG. 6A). The biopsy procedure was performed with a punch biopsy with the tissue cutting edge coated with a biocompatible dye, e.g., fluorescent dye. The dye was deposited at the exact location of the biopsy when the biopsy was performed, and was visible under UV light using Wood's lamp on the day of the biopsy (see FIG. 6B). The dye was not visible under normal white light. On the 3 month follow-up timepoint after the biopsy procedure, the subject was assessed and the location of the biopsy procedure was not visible under normal (white) light (FIG. 6C), but could be identified using illumination from a UV source, such as a Wood's lamp (FIGS. 6D and 6E). At a 6-month follow-up after the biopsy procedure, the location of the biopsy site could still be detected under illumination from a UV source, such as a Wood's lamp (data not shown), but could not be visualized under normal light. The subject reported no symptoms or side effects associates with the fluorescent tattoo, thus demonstrating an effective and safe methodology to mark and identify the location of a biopsy procedure in a subject. In fact, at the 6-month time point, the patient identified an incorrect location shown by the arrow labeled by (b) in FIG. 6F that he thought was the site of the biopsy, whereas illumination of the subjects back with UV light, e.g., Wood's lamp identified the fluorescent dye and the correct location is as shown by the arrow labeled (a) (FIG. 6F). Accordingly, the present invention demonstrates the usefulness of the system, methods and devices to correctly identify the exact location of a biopsy site at subsequent follow-up assessments following a biopsy procedure, without the biopsy mark being visible to the eye. In fact, this case study also demonstrates that the fluorescent tattoo is invisible under normal light, as had the fluorescent tattoo been visible under normal (white) light, the subject would have not mis-identified or mistaken the location of the biopsy site. The patient did not experience any symptoms or side effects with the fluorescent tattoo.

### REFERENCES

All references cited in the specification and the Examples are incorporated herein in their entirety by reference.

1. A tissue marking system comprising combining a biopsy device with a biocompatible dye, wherein the biopsy device applies the biocompatible dye to the biopsy cavity walls or surface of a subject's tissue during a biopsy procedure, and wherein the biocompatible dye is visible under a predetermined wavelength.

2. The tissue marking system of claim 1, wherein the biocompatible dye is a fluorescent dye.

3. The tissue marking system of claim 1, wherein the biocompatible dye is not visible or minimally visible under normal (white) light.

4. The tissue marking system of claim 1, wherein the biocompatible dye is visible under UV light or black light

5. The tissue marking system of claim 1, wherein the biocompatible dye is visible at under 400 nm wavelength.

6. The tissue marking system of claim 1, wherein the biocompatible dye is visible at about 365 nm wavelength.

7. The tissue marking system of claim 1, wherein the device comprises a tissue cutting edge.

8. The tissue marking system of claim 7, wherein the cutting edge is coated with the biocompatible dye.

9. The tissue marking system of claim 1, wherein the tissue is skin.

10. (canceled)

11. (canceled)

12. The tissue marking system of claim 8, wherein the biocompatible dye is semi-permanent.

13.-16. (canceled)

17. A biopsy device comprising at least one tissue cutting edge, wherein at least one tissue cutting edge is coated with a biocompatible dye, wherein the biocompatible dye is reactive under a predefined wavelength.

18.-25. (canceled)

26. The biopsy device of claim 17, wherein the biopsy device is selected from the group consisting of: needle biopsy device, hookwire biopsy device, photonic needle, clamp, forceps, micro-scissors, punch biopsy device, core biopsy device, razor, scapel blade, suture, shave biopsy device, a cutaneous needle biopsy device.

27. The biopsy device of claim 17, wherein the biopsy device is used for a punch biopsy or a shave biopsy.

28.-32. (canceled)

33. A method of determining the site of a biopsy, comprising:
a. using a biopsy device comprising at least one tissue cutting edge, wherein at least one tissue cutting edge is coated with a biocompatible dye, wherein the biocompatible dye is reactive under a predetermined wavelength, and wherein the biopsy device applies the biocompatible dye to the biopsy cavity walls or surface of a subject's tissue during an initial biopsy as it is being performed to mark the site of the biopsy;
b. locating the biopsy site at a subsequent timepoint by illuminating the skin with the predefined wavelength.

34. (canceled)

35. (canceled)

36. The method of claim 33 comprising identifying the location of the biopsy by illuminating the skin with the predetermined wavelength, wherein the illuminating occurs at a timepoint that is after the marking of the site of the biopsy.

37. (canceled)

38. The method of claim 36, wherein the illuminating is at least 2 weeks after the marking of biopsy site.

39.-41. (canceled)

42. The method of claim 36, wherein the illuminating is at least 6 months after the marking of biopsy site.

43.-46. (canceled)

47. A kit comprising a container comprising a biocompatible dye for coating a tissue cutting edge of a biopsy device.

48. The kit of claim 47, further comprising a biopsy device.

49. The kit of claim 48, wherein the biopsy device is a disposable biopsy device or a disposable attachment for a non-disposable biopsy device.

50. The kit of claim 49, wherein the disposable attachment for a non-disposable biopsy device comprises at least one tissue cutting edge.

51. The kit of claim 47, further comprising an apparatus to aid coating the tissue cutting edge of a biopsy device with the biocompatible dye.

52. The kit of claim 51, wherein the biocompatible dye is a fluorescent dye.

53.-68. (canceled)
